(19) United States
(12) Patent Application Publication
(10) Pub. No.: US 2012/0039923 A1
(43) Pub. Date: Feb. 16, 2012

(54) Modified HIV-1 Envelope Proteins

(75) Inventors: Christopher Broder, Silver Spring, MD (US); Gerald Quinnan, Rockville, MD (US)

(73) Assignee: THE HENRY M. JACKSON FOUNDATION, Rockville, MD (US)

(21) Appl. No.: 11/662,422

(22) PCT Filed: Sep. 9, 2005
(86) PCT No.: PCT/US2005/032200

§ 371 Date: Oct. 11, 2007

Related U.S. Application Data
(60) Provisional application No. 60/608,144, filed on Sep. 9, 2004.

(51) Int. Cl. A61K 39/21; C12N 15/49; A61K 38/16; C12N 15/63; A61P 31/18; C12N 1/21; C12N 1/19; C12N 5/10; C07K 16/10; C07K 14/16; C12N 1/00

(52) U.S. Cl. 424/188.1; 530/350; 536/23.72; 514/3.8; 424/208.1; 435/320.1; 435/243; 435/252.33; 435/254.2; 435/348; 435/325; 435/358; 435/357; 435/352; 530/389.4

(57) ABSTRACT

The present invention relates to modified HIV-1 envelope proteins where one or more N-glycosylation sites have been deleted or modified, which produce a broadly cross reactive neutralizing response, their methods of use and antibodies which bind to these proteins. The invention also provides for nucleic acids, vectors, antibodies and pharmaceutical compositions that comprise said modified HIV-1 envelope proteins.

### ACKNOWLEDGMENT OF FEDERAL SUPPORT

The present invention arose in part from research funded by federal grants NIH AI48380-01 and AI42599-01.

### FIELD OF THE INVENTION

The present invention relates to modified HIV-1 envelope proteins which confer the capacity to neutralize primary HIV-1 isolates of varied subtypes following immunization in a mammal.

### BACKGROUND OF THE INVENTION

Human immunodeficiency virus type-1 (HIV-1) is the etiologic agent of acquired immunodeficiency syndrome (AIDS). The HIV-1 strains that account for the global pandemic are designated the group M (major) strains, which are classified into some ten genetic subtypes or clades. The HIV-1 M group subtypes are phylogenetically associated groups of HIV-1 sequences, and are labeled A, B, C, D, F1, F2, G, H, J and K (Korber et al. (1999) Human Retroviruses and AIDS (vol. III) 492-505). The sequences within any one subtype are more similar to each other than to sequences from other subtypes throughout their genomes. These subtypes represent different lineages of HIV, and have some geographical associations. Former subtypes E and I are both now defined as circulating recombinant forms (CRF) (Korber et al. (1999) Human Retroviruses and AIDS (vol. III) 492-505). HIV-1 infection is generally characterized by a progressive and irreversible decline in the number of CD4+ lymphocytes (Pantaleo et al. (1993) N. Eng. J. Med. 328, 327-335) and an increase in the viral burden (Pantaleo et al. (1993) Nature 362, 355-358; Piatak et al. (1993) Lancet 341, 1099).

The demonstration of rapid turnover of HIV-1 in plasma suggests that there are natural mechanisms at work that can effectively mediate the clearance of virus (Ho et al. (1995) Nature 373, 123-126; Wei et al. (1995) Nature 373, 117-122). In fact, anti-HIV antibody has recently been shown to increase the clearance of virus three-fold (Igarashi et al. (1999) Nat. Med. 5, 211-216). Although active cellular and humoral immune responses to HIV infection are observed, the correlates of protective immunity remain obscure and natural infection fails to elicit a sterilizing or protective immune response. This failure of the host immune response to contain the infection, together with the complexities of viral replication, persistence, intracellular mode of transmission, mucosal port of entry, and the natural predilection of the virus for genetic change has made vaccine development a formidable task.

HIV-1 has a single transmembrane envelope glycoprotein (Env) which projects from the viral surface and infected cells. Env serves at least two functions that are critical in the life-cycle of the virus; binding to cellular receptors (CD4 and coreceptors) and mediating the fusion of viral and cellular membranes. As a consequence, the viral genome gains entry to the cytoplasm and infection can proceed. The ultimate goal in vaccine development for HIV-1 is to prevent infection, but vaccine-induced modification of HIV-1-mediated disease would also be an important advance. Traditionally, the antibody response has been the immunologic measure of vaccine efficacy. Indeed, antibody is the only vaccine-inducible effector mechanism that could prevent the infection of the initial host cell, potentially mediate the lysis of virus-infected cells by antibody-dependent cell-mediated cytolysis, and prevent cell-cell transmission through the specific interference of Env-mediated fusion. Env is also the major antigenic target for virus-neutralizing antibodies, and therefore it is theoretically conceivable that an efficacious vaccine based, at least in part, on purified Env components can formulated. Indeed, passive protection by antibodies against a low-dose, intravenous, cell-free HIV challenge has been reported (Koup et al. (1996) Semin. Immunol. 8, 263-268; Parren et al. (1995) AIDS 9, F1-F6; Parren et al. (2001) J. Virol. 75, 8340-8347; Prince et al. (1991) AIDS Res. Hum. Retroviruses 7, 971-973) and a number of studies have implicated humoral responses to Env in protection (Berman et al. (1990) Nature 345, 622-625; Berman et al. (1996) J. Infect. Dis. 173, 52-59; Girard et al. (1995) J. Virol. 69, 6239-6248).

Recently, anti-Env antibody has been shown to mediate complete protection in a macaque-SHIV model as well (Shibata et al. (1999) Nat. Med. 5, 204-210). Unfortunately, the humoral response to candidate Env vaccine preparations thus far has been largely type specific, and do not possess adequate neutralizing activity towards divergent strains, notably, primary field isolates. An ideal Env-based vaccine preparation should elicit both type specific and broadly neutralizing antibodies to a variety of antigenic determinants. The development of broadly reactive neutralizing antibodies should be possible, and several studies have shown that serum from HIV-1 infected individuals contains a high proportion of broadly neutralizing antibody reactivity (Ho et al. (1992) AIDS Res. Hum. Retroviruses 8, 1337-1339; Moore et al. (1994) J. Virol. 68, 5142-5155; Moore et al. (1993) J. Virol. 67, 863-887; Steimer et al. (1991) Science 254, 105-108). These kinds of antibodies are almost invariably reactive to conformation-dependent epitopes in the Env glycoprotein: their ability to recognize Env is based on the molecule's tertiary structure. This is in contrast to most type specific antibodies that recognize conformation-independent (linear) epitopes: they react with denatured Env protein as well as the correctly folded molecule.

HIV-1 Env is a complex oligomer comprised of multiple gp120 and gp41 subunits, and evidence to date indicates that the native Env oligomer is trimer (Center et al. (2002) J. Virol. 76, 7863-7867; Center et al. (2001) Proc. Natl. Acad. Sci. USA 98, 14877-14882; Lu et al. (1995) Nat. Struct. Biol. 2, 1075-1082; Lu et al. (1997) J. Biomol. Struct. Dyn. 15, 465-471). The present invention focuses on modified Env protein as a candidate vaccine immunogen. Previously it was found that immunization with soluble, oligomeric gp140 derived from the IIIB isolate (gp140_{IIIB}) effectively generated a more broadly cross-reactive antibody response as compared to immunization with monomeric Env. It was also determined that immunization with gp140 could elicit a wide variety of monoclonal antibody (MAb) reactivity, some of which where highly specific for Env tertiary structure and broadly cross-reactive, and possessed weak neutralizing activity. These monoclonal antibodies mapped to cluster I of the gp41 ectodomain (FIG. 1).

### SUMMARY OF THE INVENTION

The invention encompasses a modified envelope protein or fragment thereof comprising one or more modifications at one or more N-glycosylations sites which, when administered to a mammal, induces the production of broadly cross-reactive neutralizing anti-serum against multiple subtypes of HIV-1. In some embodiments, the glycosylation sites are deleted, while in other embodiments they are substituted with another amino acid other than asparginine, such as glutamine or any other conservative substitution. In one embodiment, the modified HIV-1 envelope protein is derived from gp160, gp140, gp120 and/or gp41 or fragment thereof. In another embodiment, the modified HIV-1 envelope protein is an oligomeric HIV-1 envelope protein. In another embodiment, the modified oligomeric HIV-1 envelope protein is derived from gp160, gp120 and/or gp41 or a fragment thereof. In another embodiment, the modified oligomeric HIV-1 envelope protein is gp140 envelope protein or fragment thereof. The modified HIV-1 envelope protein will comprise one or more modifications at one or more N-glycosylations sites which, when administered to a mammal, induces the production of a broadly cross-reactive neutralizing anti-serum against multiple subtypes of HIV-1. The modified N-glycosylation sites are selected from one or more amino acids corresponding to residues 610, 615, 624 and 636 of SEQ ID NO: 2, 4, 6, 8, 10, 12, 14 and 16. In one embodiment, the modified gp140 comprises and/or consists of a sequence selected from the group consisting of SEQ ID NO: 2, 4, 6, 8, 10, 12, 14 and 16.

The invention also encompasses nucleic acid molecules encoding the modified HIV-1 gp160, gp140, gp120 and/or gp41 envelope protein or fragment thereof described herein. In one embodiment, the modified envelope nucleic acid molecule comprises and/or consists of SEQ ID NO: 1, 3, 5, 7, 9, 11, 13 and 15 or fragments thereof. The isolated nucleic acid molecule of the invention may be operably linked to one or more expression control elements. The invention also includes a vector comprising the isolated nucleic acid molecule of the invention, a host cell containing this vector and methods for the recombinant expression of the modified HIV-1 envelope protein.

The invention further includes a composition comprising the modified HIV-1 envelope protein or fragment thereof as described herein and a pharmaceutically acceptable carrier. In one embodiment, the pharmaceutical composition comprises one or more modified HIV-1 envelope protein from the group consisting of gp160, gp140, gp120, and gp41. In another embodiment, the pharmaceutical composition comprises one or more modified oligomeric HIV-1 envelope protein. In another embodiment, the modified oligomeric HIV-1 envelope protein is derived from gp160, gp120 and/or gp41 or fragment thereof. In another embodiment, the modified oligomeric HIV-1 envelope protein is gp140 envelope protein or fragment thereof. In another embodiment, the composition is suitable as a vaccine in humans.

The invention yet further includes fusion proteins comprising a modified HIV-1 envelope protein or fragment thereof linked to at least one second protein. In one embodiment, fusion proteins of the invention comprise a modified HIV-1 envelope protein from the group consisting of gp160, gp140, gp120, and gp41. In another embodiment, the fusion protein of the invention comprises a modified oligomeric HIV-1 envelope protein. In another embodiment, the fusion protein comprises an oligomeric gp160, gp140, gp120, and/or gp41 fused to a second protein.

The invention also includes a method of generating antibodies in a mammal comprising administering one or more of the modified HIV-1 envelope proteins or fragments thereof in an amount sufficient to induce the production of the antibodies. In some embodiments, the HIV-1 gp160, gp140, gp120 and/or gp41 envelope protein or fragment thereof, when administered to a mammal, induces the production of broadly cross-reactive neutralizing anti-serum against multiple strains of HIV-1. The invention further encompasses an isolated antibody produced by this method and/or which specifically binds to any one of the HIV-1 envelope proteins or fragments thereof described herein.

### BRIEF DESCRIPTION OF THE DRAWINGS

FIG. 1: Epitope structure of the ectodomain of gp41. This 2-dimensional depiction is modified from Gallaher et al. (1989) AIDS Res Hum Retroviruses 5, 431-440. The two alpha-helical regions shown to interact by Wild et al. (1994) Proc. Natl. Acad. Sci. USA 91, 12676-12680 are juxtaposed. Antigenic determinants defined by us are circled and shaded. The 2F5 epitope is circled with a bold line. The linear monoclonal antibodies used to define each group is given in parentheses. The four conserved N-glycosylation sites are indicated by 3-prong sticks in domains VI and I.

FIG. 2: Expression and processing of CM243 gp140 wild-type and glycosylation mutants. Protein were expressed and metabolically labeled with ³⁵S-methionine/cysteine. Following expression, the CM243 gp140 in the culture supernatant were immunoprecipitated by anti-gp41 monoclonal antibody D61. Large arrows point to gp41 ectodomains. Wild type CM243, single mutant N610Q and N615Q were processed to gp120 and gp41 subunits. Where processing was evident, the single N610Q resulted in the largest shift in apparent molecular weight of the gp41 ectodomain, an approximately 5 to 6 kDa loss.

FIG. 3: Reactivity of CM243 gp140 with a panel of conformation-dependent and -independent monoclonal antibodies. While equivalent reactivities with monoclonal antibodies D19, T50, D38, and T3 were seen in both wild type and mutant gp140, differential reactivities were found with monoclonal antibodies that map to the Cluster I region in gp41 (T4, T6, T9, and T10) which are conformation dependent, oligomeric specific anti-gp41 antibodies.

FIG. 4: CM243 wild type and glycosylation mutant gp140 undergo CD4-induced conformational change. Binding of CD41 antibodies 23e, 48d and 17b to both wild type and mutant CM243 gp140 increased after CD4 binding.

FIG. 5: Neutralizing antibody responses of New Zealand white rabbits immunized with gp140_{CM243} or gp140_{CM243(N610Q)} in RIBI adjuvant. Results shown are means and standard deviations for three animals per group. Neutralization was measured in a pseudotyped virus reporter assay. Upper panel shows activity in sera from animals immunized with unmodified gp140. Lower panels show activity in animals immunized with the N610Q mutant against CM243 and GX-#14 strains of HIV-1.

FIG. 6: Neutralization of subtype C, B, and D pseudotyped viruses by sera from gp140_{CM243} and gp140_{CM243(N610Q)} immunized rabbits. Results shown for individual rabbits are 50% inhibition endpoints. The 90% endpoints are about four-fold lower. Open circles: prebleeds; Closed circles: post-immune.

FIG. 7: Alignment of different HIV-1 envelope proteins.

### DETAILED DESCRIPTION

A goal of immunization against HIV is to induce neutralizing antibody (NA) responses broadly reactive against diverse strains of virus. The present inventors have studied HIV-1 envelope protein and determined that modification of one or more glycosylation sites in this protein induces the production of broadly cross-reactive species of antibodies following immunization. The invention therefore encompasses the HIV-1 envelope proteins with modifications at the glycosylation sites, methods of use, and antibodies generated against these proteins.

Thus, in accordance with the present invention, there are provided methods for inhibiting, preventing, and ameliorating a viral infection in a subject. In one embodiment, a method of the invention includes administering an effective amount of an antibody that binds to a modified HIV-1 envelope protein to a subject, thereby preventing or inhibiting virus infection in the subject. In another embodiment, a method of the invention includes administering an effective amount of a modified HIV-1 envelope protein to a subject, thereby producing an immune response sufficient for preventing or inhibiting virus infection in the subject. In yet another embodiment, a method of the invention includes administering to a subject an effective amount of a nucleic acid encoding a modified HIV-1 envelope protein.

### Modified Envelope Proteins

The invention encompasses HIV-1 gp160, gp140 and gp120 envelope proteins which are modified in the gp41 ectodomain with respect to a wild type (native) HIV-1 gp41 in the primary amino acid sequence to effect whole or partial deglycosylation. Potential N-glycosylation sites, preferably in the gp41 ectodomain, can be systematically modified, either singly or in combination by site directed mutagenesis such that the consensus glycosylation sequence is disrupted. There are generally four potential N-glycosylation sites in gp41, the present invention encompasses modification of at least one, two, three or four of these sites in any potential combination in any potential manner. Notwithstanding the mutation(s), the conformation of the envelope protein remains sufficiently intact to maintain infectivity when present as a component of the virion. Individuals (i.e., humans) that are immunized with this modified proteins develop an immune response which will reduce or block viral infectivity.

Modified gp160, gp140, gp120 and/or gp41 envelope proteins of the invention include the full length envelope protein wherein one or more N-glycosylation sites have been modified and fragments thereof containing one or more of the modified N-glycosylation sites. In one embodiment, one or more N-glycosylation sites are deleted while in another embodiment, one or more of these sites are substituted with another amino acid which is not capable of being glycosylated. Examples of amino acid which are not capable of glycosylation include, but are not limited to, any naturally occurring amino acid other than asparginine. Preferred naturally occurring amino acids which can be substituted include, but are not limited to, glutamine. Modified amino acids can also be used as substitutes at any N-glycosylation sites. Such modified amino acids are incapable of being glycosylated.

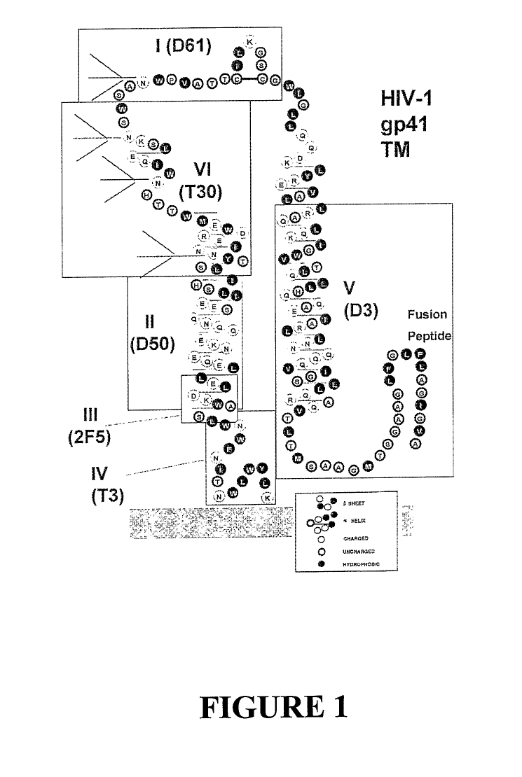

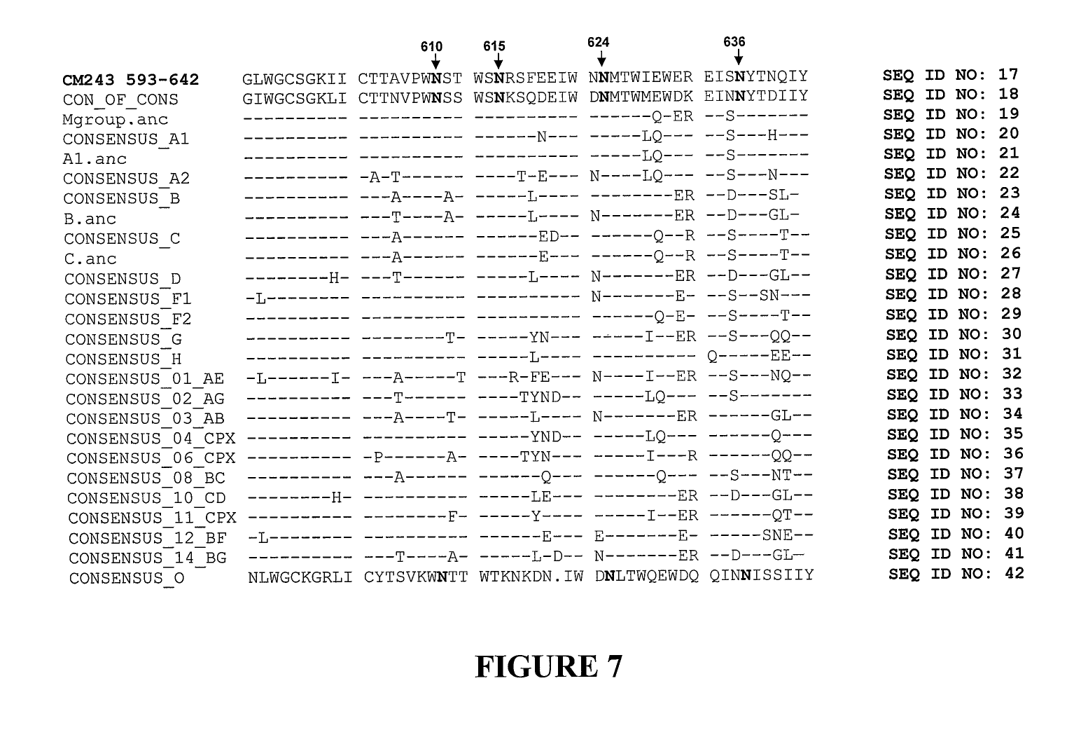

In general, there are four consensus N-glycosylation sites in the gp41 coding sequence of HIV-1 isolates. For illustrative purposes, the positions of these sites on gp41 in CM243 (clade E, R5 primary isolate) are shown in FIG. 7. The relative positions of these sites on the predicted structure of gp41 in CM243 are also shown (FIG. 1). Amino acid and nucleotide sequence information for envelope proteins of other strains are referenced in Kuiken et al. (2002) HIV Sequence Compendium, Los Alamos National Laboratory, LA-UR03-3564, which is hereby incorporated by reference. Exemplary N-glycosylation sites in the gp41 envelope protein include, but are not limited to, amino acids in gp41 envelope proteins corresponding to residues 610, 615, 624 and 636 of SEQ ID NO: 2, 4, 6, 8, 10, 12, 14 and 16. As illustrated in FIG. 7, the alignment of envelope sequences of different clades show a conserved asparagine at residue positions 610, 615, 624 and 636. In addition, the corresponding residues which are N-glycosylation sites in gp41 envelope proteins from other HIV-1 isolates, which may not have the same residue number, can readily be determined by amino acid sequence alignment as set forth herein (see FIG. 7). Thus, in another embodiment, the invention encompasses modification of envelope proteins of different clades of HIV-1. For example, the invention encompasses modifications of envelope protein of clades M (for main), N (for non-M/non-O), and O (for outlier) as well as Glade subtypes. For example, Glade M subtypes are labeled A, B, C, D, F1, F2, G, H, J and K (Korber et al. (1999) Human Retroviruses and AIDS (vol. III) 492-505).

In another embodiment, the invention encompasses oligomeric gp140 envelope proteins comprising the amino acid sequence as set forth in SEQ ID NO: 2, 4, 6, 8, 10, 12, 14 and 16 and fragments thereof containing one or more of the modified N-glycosylation sites. In yet another embodiment, the invention encompasses oligomeric gp140 envelope proteins consisting of the amino acid sequence as set forth in SEQ ID NO: 2, 4, 6, 8, 10, 12, 14 and 16. In another embodiment, the invention encompasses modification of the N-glycosylation sites of the gp160 and gp120 in their corresponding amino acid residues.

Although not wishing to be bound by theory, the invention contemplates that hidden epitopes (cryptic epitopes) will be exposed when the N-glycosylation site are modified and/or deleted. It is contemplated that modifying one or more N-glycosylation site will expose a cryptic epitope either at or near the site of the modification or as a result of an overall conformation change due to the aforementioned modification(s). Therefore, it is envisaged that modified HIV-1 envelope proteins of the invention expose one or more cryptic epitopes that can lead to a broad neutralization of HIV-1 when administered to a mammal. Such epitopes may be shared among different viral isolates and geographic clades accounting for broad-spectrum neutralizing activity of the antibodies directed to these epitopes.

### Nucleic Acid Molecules

The present invention further provides nucleic acid molecules that encode the modified HIV-1 envelope proteins or fragments thereof that contain one or more of the modified N-glycosylation sites, preferably in isolated form. As used herein, “nucleic acid” is defined as RNA or DNA that encodes a protein or peptide as defined above, is complementary to a nucleic acid sequence encoding such peptides, hybridizes to nucleic acid molecules that encode the modified HIV-1 envelope proteins across the open reading frame under appropriate stringency conditions, or encodes a polypeptide that shares at least about 75% sequence identity, preferably at least about 80%, more preferably at least about 85%, and even more preferably at least about 90% or even 95% or more identity with the modified HIV-1 gp160, gp140, gp120 and/or gp41 envelope proteins.

The nucleic acids of the invention further includes nucleic acid molecules that share at least 80%, preferably at least about 85%, and more preferably at least about 90% or 95% or more identity with the nucleotide sequence of nucleic acid molecules that encode the modified HIV-1 envelope proteins, particularly across the contiguous open reading frame. Specifically contemplated are genomic DNA, cDNA, mRNA and antisense molecules, as well as nucleic acids based on alternative backbones or including alternative bases whether derived from natural sources or synthesized. Such nucleic acids, however, are defined further as being novel and unobvious over any prior art nucleic acid including that which encodes, hybridizes under appropriate stringency conditions, or is complementary to nucleic acid encoding a protein according to the present invention.

Homology or identity at the nucleotide or amino acid sequence level is determined by BLAST (Basic Local Alignment Search Tool) analysis using the algorithm employed by the programs blastp, blastn, blastx, tblastn and tblastx (Altschul et al. (1997) Nucleic Acids Res. 25, 3389-3402 and Karlin et al. (1990) Proc. Natl. Acad. Sci. USA 87, 2264-2268, both fully incorporated by reference) which are tailored for sequence similarity searching. The approach used by the BLAST program is to first consider similar segments, with and without gaps, between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. (1994) Nature Genetics 6, 119-129 which is fully incorporated by reference. The search parameters for histogram, descriptions, alignments, expect (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix (Henikoff et al. (1992) Proc. Natl. Acad. Sci. USA 89, 10915-10919, fully incorporated by reference), recommended for query sequences over 85 in length (nucleotide bases or amino acids).

For blastn, the scoring matrix is set by the ratios of M (i.e., the reward score for a pair of matching residues) to N (i.e., the penalty score for mismatching residues), wherein the default values for M and N are +5 and -4, respectively. Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

“Stringent conditions” are those that (1) employ low ionic strength and high temperature for washing, for example, 0.015 M NaCl/0.0015 M sodium citrate/0.1% SDS at 50° C. to 68° C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer (pH 6.5) with 750 mM NaCl, 75 mM sodium citrate at 42° C. Another example is hybridization in 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC and 0.1% SDS or 68° C. in 0.1×SSC and 0.5% SDS. A skilled artisan can readily determine and vary the stringency conditions appropriately to obtain a clear and detectable hybridization signal. Preferred molecules are those that hybridize under the above conditions to the complement of SEQ ID NO: 1, 3, 5, 7, 9, 11, 13 and 15 and which encode a functional protein. Even more preferred hybridizing molecules are those that hybridize under the above conditions to the complement strand of the open reading frame of the nucleic acid encoding the modified HIV-1 envelope protein. As used herein, a nucleic acid molecule is said to be “isolated” when the nucleic acid molecule is substantially separated from contaminant nucleic acid molecules encoding other polypeptides.

The present invention further provides fragments of the encoding nucleic acid molecule which contain the desired modification (i.e., modification of one or more N-glycosylation sites) in the envelope proteins. As used herein, a fragment of an encoding nucleic acid molecule refers to a small portion of the entire protein coding sequence. The size of the fragment will be determined by the intended use. For example, if the fragment is chosen so as to encode an active portion of the protein (i.e., a modified N-glycosylation site), the fragment will need to be large enough to encode the functional regions of the protein (i.e., epitopes). For instance, fragments which encode peptides corresponding to predicted antigenic regions may be prepared. If the fragment is to be used as a nucleic acid probe or PCR primer, then the fragment length is chosen so as to obtain a relatively small number of false positives during probing/priming.

Fragments of the encoding nucleic acid molecules of the present invention (i.e., synthetic oligonucleotides) that are used to synthesize gene sequences encoding proteins of the invention, can easily be synthesized by chemical techniques, for example, the phosphotriester method of Matteucci et al. (1981) J. Am. Chem. Soc. 103, 3185-3191 or using automated synthesis methods. In addition, larger DNA segments can readily be prepared by well known methods, such as synthesis of a group of oligonucleotides that define various modular segments of the gene, followed by ligation of oligonucleotides to build the complete modified gene. In a preferred embodiment, the nucleic acid molecule of the present invention contains a contiguous open reading frame of at least about three-thousand and forty-five nucleotides.

The encoding nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can readily employ any such label to obtain labeled variants of the nucleic acid molecules of the invention. Modifications to the primary structure itself by deletion, addition, or alteration of the amino acids incorporated into the protein sequence during translation can be made without destroying the activity of the protein. Such substitutions or other alterations result in proteins having an amino acid sequence encoded by a nucleic acid falling within the contemplated scope of the present invention.

### Recombinant Nucleic Acids

The present invention further provides recombinant DNA molecules (rDNA) that contain a coding sequence. As used herein, a rDNA molecule is a DNA molecule that has been subjected to molecular manipulation in situ. Methods for generating rDNA molecules are well known in the art, for example, see Sambrook et al. (2001) Molecular Cloning—A Laboratory Manual, Cold Spring Harbor Laboratory Press. In the preferred rDNA molecules, a coding DNA sequence is operably linked to expression control sequences and/or vector sequences.

The choice of vector and/or expression control sequences to which one of the protein family encoding sequences of the present invention is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 (BioRad), pPL and pKK223 (Pharmacia).

Expression vectors compatible with eukaryotic cells, preferably those compatible with vertebrate cells, can also be used to form rDNA molecules that contain a coding sequence. Eukaryotic cell expression vectors, including viral vectors, are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies Inc.), pTDT1 (ATCC), the vector pCDM8 described herein, and the like eukaryotic expression vectors.

Eukaryotic cell expression vectors used to construct the rDNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene. (Southern et al. (1982) J. Mol. Anal. Genet. 1, 327-341). Alternatively, the selectable marker can be present on a separate plasmid, and the two vectors are introduced by co-transfection of the host cell, and selected by culturing in the appropriate drug for the selectable marker. The present invention further provides host cells transformed with a nucleic acid molecule that encodes a protein of the present invention. The host cell can be either prokaryotic or eukaryotic.

Eukaryotic cells useful for expression of a protein of the invention are not limited, so long as the cell line is compatible with cell culture methods and compatible with the propagation of the expression vector and expression of the gene product. Preferred eukaryotic host cells include, but are not limited to, yeast, insect and mammalian cells, preferably vertebrate cells such as those from a mouse, rat, monkey or human cell line. Preferred eukaryotic host cells include Chinese hamster ovary (CHO) cells available from the ATCC as CCL61, NIH Swiss mouse embryo cells (NIH-3T3) available from the ATCC as CRL 1658, baby hamster kidney cells (BHK), and the like eukaryotic tissue culture cell lines. Any prokaryotic host can be used to express a rDNA molecule encoding a protein of the invention. The preferred prokaryotic host is E. coli.

Transformation of appropriate cell hosts with a rDNA molecule of the present invention is accomplished by well known methods that typically depend on the type of vector used and host system employed. With regard to transformation of prokaryotic host cells, electroporation and salt treatment methods are typically employed, see, for example, Cohen et al. (1972) Proc. Natl. Acad. Sci. USA 69, 2110; and Sambrook et al. (2001) Molecular Cloning—A Laboratory Manual, Cold Spring Harbor Laboratory Press. With regard to transformation of vertebrate cells with vectors containing rDNA, electroporation, cationic lipid or salt treatment methods are typically employed, see, for example, Graham et al. (1973) Virol. 52, 456; Wigler et al. (1979) Proc. Natl. Acad. Sci. USA 76, 1373-1376.

Successfully transformed cells, i.e., cells that contain a rDNA molecule of the present invention, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern (1975) J. Mol. Biol. 98, 503-504 or Berent et al. (1985) Biotech. 3, 208-209 or the proteins produced from the cell assayed via an immunological method.

### Production of Recombinant Proteins

One skilled in the art would know how to make recombinant nucleic acid molecules which encode the modified HIV-1 envelope proteins of the invention using both in vitro and in vivo systems. Furthermore, one skilled in the art would know how to use these recombinant nucleic acid molecules to obtain the proteins encoded thereby, as described herein for the recombinant nucleic acid molecule which encodes a modified HIV-1 envelope protein comprising one or more modifications at one or more N-glycosylation sites.

In accordance with the invention, numerous vector systems for expression of the modified HIV-1 envelope protein may be employed. For example, one class of vectors utilizes DNA elements which are derived from animal viruses such as bovine papilloma virus, polyoma virus, adenovirus, vaccinia virus, baculovirus, retroviruses (RSV, MMTV or MoMLV), Semliki Forest virus or SV40 virus. Additionally, cells which have stably integrated the DNA into their chromosomes may be selected by introducing one or more markers which allow for the selection of transfected host cells. The marker may provide, for example, prototropy to an auxotrophic host, biocide resistance, (e.g., antibiotics) or resistance to heavy metals such as copper or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals. The cDNA expression vectors incorporating such elements include those described by Okayama (1983) Mol. Cell. Biol. 3, 280-289.

The vectors used in the subject invention are designed to express high levels of modified HIV-1 envelope proteins in cultured eukaryotic cells as well as efficiently secrete these proteins into the culture medium. In one embodiment, the targeting of the envelope glycoproteins into the culture medium is accomplished by fusing in-frame to the mature N-terminus of the modified gp160, gp140, gp120 and/or gp41 envelope protein in the tissue plasminogen activator (tPA) prepro-signal sequence.

The modified envelope protein may be produced by (a) transfecting a mammalian cell with an expression vector encoding the modified gp160, gp140, gp120 and/or gp41 envelope protein; (b) culturing the resulting transfected mammalian cell under conditions such that modified envelope protein is produced; and (c) recovering the modified envelope protein from the cell culture media or the cells themselves.

Once the expression vector or DNA sequence containing the constructs has been prepared for expression, the expression vectors may be transfected or introduced into an appropriate mammalian cell host. Various techniques may be employed to achieve this, such as, for example, protoplast fusion, calcium phosphate precipitation, electroporation or other conventional techniques. In the case of protoplast fusion, the cells are grown in media and screened for the appropriate activity.

Methods and conditions for culturing the resulting transfected cells and for recovering the modified envelope protein so produced are well known to those skilled in the art, and may be varied or optimized depending upon the specific expression vector and mammalian host cell employed.

In accordance with the claimed invention, the preferred host cells for expressing the modified envelope glycoprotein of this invention are mammalian cell lines. Mammalian cell lines include, for example, monkey kidney CV1 line transformed by SV40 (COS-7); human embryonic kidney line 293 (HEK293); baby hamster kidney cells (BHK); Chinese hamster ovary-cells-DHFR (CHO); Chinese hamster ovary-cells DHFR(DXB11); monkey kidney cells (CV1); African green monkey kidney cells (VERO-76); human cervical carcinoma cells (HELA); canine kidney cells (MDCK); human lung cells (W138); human liver cells (HepG2); mouse mammary tumor (MMT 060562); mouse cell line (C127); and myeloma cell lines.

Other eukaryotic expression systems utilizing non-mammalian vector/cell line combinations can be used to produce the modified envelope proteins. These include, but are not limited to, baculovirus vector/insect cell expression systems and yeast shuttle vector/yeast cell expression systems.

Methods and conditions for purifying modified envelope proteins from the culture media are provided in the invention, but it should be recognized that these procedures can be varied or optimized as is well known to those skilled in the art.

The invention encompasses methods for producing the modified envelope proteins utilizing in vivo expression systems in mammals which are well known in the art. Such mammals include, but are not limited to, humans, cows, sheep, pigs, etc. Also contemplated are alpha virus gene vectors that can be employed to produce the modified envelope proteins. Preferred alpha virus vectors are Sindbis viruses vectors. Also contemplated are togaviruses, Semliki Forest virus, Middleberg virus, Ross River virus, Venezuelan equine encephalitis virus and those described in U.S. Pat. Nos. 5,091,309 and 5,217,879. More particularly, those alpha virus vectors described in WO94/21792, WO92/10578, WO95/07994, U.S. Pat. Nos. 5,091,309 and 5,217,879. Such alpha viruses may be obtained from commercial sources or isolated from known sources using commonly available techniques.

DNA vector systems such as eukaryotic layered expression systems are also useful for expressing the modified envelope proteins. See WO95/07994 for a detailed description of eukaryotic layered expression systems. Preferably, the eukaryotic layered expression systems of the invention are derived from alpha virus vectors and most preferably from Sindbis viral vectors.

The modified envelope proteins of the present invention may also be prepared by any known synthetic techniques. Conveniently, the proteins may be prepared using the solid-phase synthetic technique initially described by Merrifield (1965), which is incorporated herein by reference. Other peptide synthesis techniques may be found, for example, in Bodanszky et al. (1976), Peptide Synthesis, Wiley.

### Modified Envelope Fusion Proteins

Modified, envelope fusion proteins and methods for making such proteins have been previously described (U.S. Pat. Nos. 6,171,596 and 6,039,957). It is now a relatively straight forward technology to prepare cells expressing a foreign gene. Such cells act as hosts and may include, for the fusion proteins of the present invention, yeasts, fungi, insect cells, plants cells or animals cells. Expression vectors for many of these host cells have been isolated and characterized, and are used as starting materials in the construction, through conventional recombinant DNA techniques, of vectors having a foreign DNA insert of interest. Any DNA is foreign if it does not naturally derive from the host cells used to express the DNA insert. The foreign DNA insert may be expressed on extrachromosomal plasmids or after integration in whole or in part in the host cell chromosome(s), or may actually exist in the host cell as a combination of more than one molecular form. The choice of host cell and expression vector for the expression of a desired foreign DNA largely depends on availability of the host cell and how fastidious it is, whether the host cell will support the replication of the expression vector, and other factors readily appreciated by those of ordinary skill in the art.

The foreign DNA insert of interest comprises any DNA sequence coding for fusion proteins including any synthetic sequence with this coding capacity or any such cloned sequence or combination thereof. For example, fusion proteins coded and expressed by an entirely recombinant DNA sequence is encompassed by this invention but not to the exclusion of fusion proteins peptides obtained by other techniques.

Vectors useful for constructing eukaryotic expression systems for the production of fusion proteins comprise the fusion protein's DNA sequence, operatively linked thereto with appropriate transcriptional activation DNA sequences, such as a promoter and/or operator. Other typical features may include appropriate ribosome binding sites, termination codons, enhancers, terminators, or replicon elements. These additional features can be inserted into the vector at the appropriate site or sites by conventional splicing techniques such as restriction endonuclease digestion and ligation.

Yeast expression systems, which are the preferred variety of recombinant eukaryotic expression system, generally employ Saccharomyces cerevisiae as the species of choice for expressing recombinant proteins. Other species of the genus Saccharomyces are suitable for recombinant yeast expression system, and include but are not limited to carlsbergensis, uvarum, rouxii, montanus, kluyveri, elongisporus, norbensis, oviformis, and diastaticus. Saccharomyces cerevisiae and similar yeasts possess well known promoters useful in the construction of expression systems active in yeast, including but not limited to GAP, GAL10, ADH2, PHO5, and alpha mating factor.

Yeast vectors useful for constructing recombinant yeast expression systems for expressing fusion proteins include, but are not limited to, shuttle vectors, cosmid plasmids, chimeric plasmids, and those having sequences derived from two micron circle plasmids. Insertion of the appropriate DNA sequence coding for fusion proteins into these vectors will, in principle, result in a useful recombinant yeast expression system for fusion proteins where the modified vector is inserted into the appropriate host cell, by transformation or other means. Recombinant mammalian expression system are another means of producing the fusion proteins for the vaccines/immunogens of this invention. In general, a host mammalian cell can be any cell that has been efficiently cloned in cell culture. However, it is apparent to those skilled in the art that mammalian expression options can be extended to include organ culture and transgenic animals. Host mammalian cells useful for the purpose of constructing a recombinant mammalian expression system include, but are not limited to, Vero cells, NIH3T3, GH3, COS, murine C127 or mouse L cells. Mammalian expression vectors can be based on virus vectors, plasmid vectors which may have SV40, BPV or other viral replicons, or vectors without a replicon for animal cells. Detailed discussions on mammalian expression vectors can be found in the treatises of Glover (1985), DNA Cloning: A Practical Approach, IRL Press.

Fusion proteins may possess additional and desirable structural modifications not shared with the same organically synthesized peptide, such as adenylation, carboxylation, N- and O-glycosylation, hydroxylation, methylation, phosphorylation or myristylation. These added features may be chosen or preferred as the case may be, by the appropriate choice of recombinant expression system. On the other hand, fusion proteins may have its sequence extended by the principles and practice of organic synthesis.

### Vaccine Compositions

When used in vaccine or immunogenic compositions, the modified gp160, gp140, gp120 and/or gp41 envelopes proteins of the present invention may be used as “subunit” vaccines or immunogens. Such vaccines or immunogens offer significant advantages over traditional vaccines in terms of safety and cost of production; however, subunit vaccines are often less immunogenic than whole-virus vaccines, and it is possible that adjuvants with significant immunostimulatory capabilities may be required in order to reach their full potential.

Currently, adjuvants approved for human use in the United States include aluminum salts (alum). These adjuvants have been useful for some vaccines including hepatitis B, diphtheria, polio, rabies, and influenza. Other useful adjuvants include Complete Freund's Adjuvant (CFA), Incomplete Freund's Adjuvant (IFA), Muramyl dipeptide (MDP), synthetic analogues of MDP, N-acetylmuramyl-L-alanyl-D-isoglutamyl-L-alanine-2-[1,2-d]palmitoyl-s-glycero-3-(hydroxyphosphoryloxy)]ethylamide (MTP-PE) and compositions containing a degradable oil and an emulsifying agent, wherein the oil and emulsifying agent are present in the form of an oil-in-water emulsion having oil droplets substantially all of which are less than one micron in diameter.

The formulation of a vaccine or immunogenic compositions of the invention will employ an effective amount of the protein or peptide antigen. That is, there will be included an amount of antigen which, in combination with the adjuvant, will cause the subject to produce a specific and sufficient immunological response so as to impart protection to the subject from subsequent exposure to HIV. When used as an immunogenic composition, the formulation will contain an amount of antigen which, in combination with the adjuvant, will cause the subject to produce specific antibodies which may be used for diagnostic or therapeutic purposes.

The vaccine compositions of the invention may be useful for the prevention or therapy of HIV-1 infection. While all animals that can be afflicted with HIV-1 or their equivalents can be treated in this manner, the invention, of course, is particularly directed to the preventive and therapeutic use of the vaccines of the invention in humans. Often, more than one administration may be required to bring about the desired prophylactic or therapeutic effect; the exact protocol (dosage and frequency) can be established by standard clinical procedures.

The vaccine compositions are administered in any conventional manner which will introduce the vaccine into the animal, usually by injection. For oral administration the vaccine composition can be administered in a form similar to those used for the oral administration of other proteinaceous materials. As discussed above, the precise amounts and formulations for use in either prevention or therapy can vary depending on the circumstances of the inherent purity and activity of the antigen, any additional ingredients or carriers, the method of administration and the like.

By way of non-limiting illustration, the vaccine dosages administered will typically be, with respect to the antigen, a minimum of about 0.1 mg/dose, more typically a minimum of about 1 mg/dose, and often a minimum of about 10 mg/dose. The maximum dosages are typically not as critical. Usually, however, the dosage will be no more than 500 mg/dose, often no more than 250 mg/dose. These dosages can be suspended in any appropriate pharmaceutical vehicle or carrier in sufficient volume to carry the dosage. Generally, the final volume, including carriers, adjuvants, and the like, typically will be at least 0.1 ml, more typically at least about 0.2 ml. The upper limit is governed by the practicality of the amount to be administered, generally no more than about 0.5 ml to about 1.0 ml.

In an alternative format, vaccine or immunogenic compositions may be prepared as vaccine vectors which express the modified gp160, gp140, gp120 and/or gp41 envelope protein or fragment thereof in the host animal. Any available vaccine vector may be used, including live Venezuelan Equine Encephalitis virus (see U.S. Pat. No. 5,643,576), poliovirus (see U.S. Pat. No. 5,639,649), pox virus (see U.S. Pat. No. 5,770,211) and vaccina virus (see U.S. Pat. Nos. 4,603,112 and 5,762,938). Alternatively, naked nucleic acid encoding the protein or fragment thereof may be administered directly to effect expression of the antigen (see U.S. Pat. No. 5,739,118).

### Antibodies and Methods of Use

This invention further provides a human monoclonal antibody directed to an epitope on the modified envelope glycoproteins of the invention and capable of blocking the binding of HIV-1 to human cells and capable preventing infection of human cells by HIV-1 both in vitro and in vivo. In one embodiment of the invention, the epitope recognized by the human monoclonal antibody is one of the epitopes defined in FIG. 1, including, but not limited to, any epitope which contains one or more modifications to an N-glycosylation site in the gp41 ectodomain. This invention also provides the human monoclonal antibodies.

Although not wishing to be bound by theory, the invention contemplates that hidden epitopes (cryptic epitopes) that will be exposed when the N-glycosylation site are modified and/or deleted. It is contemplated that modifying one or more N-glycosylation site will expose a cryptic epitope either at or near the site of the modification or as a result of an overall conformation change due to the aforementioned modification(s). Thus, in accordance with the present invention, antibodies that bind to modified envelope proteins of the invention, including antibodies specific for cryptic epitopes exposed upon modification of the envelope protein as set forth herein, are provided. In one embodiment, the antibody neutralizes multiple viral isolates and viruses from different geographic clades (termed “broadly neutralizing”) in vitro. In another embodiment, the antibody inhibits, prevents, or blocks virus infection in vitro or in vivo. Antibody comprising polyclonal antibodies, pooled monoclonal antibodies with different epotopic specificities, and distinct monoclonal antibody preparations, also are provided.

The monoclonal antibodies of the invention may be labeled with a detectable marker. Detectable markers useful in the practice of this invention are well known to those of ordinary skill in the art and may be, but are not limited to radioisotopes, dyes or enzymes such as peroxidase or alkaline phosphatase. In addition, the monoclonal antibodies of the invention may be conjugated with a cytotoxic agent.

This invention also concerns an anti-idiotypic antibody directed against the human monoclonal antibodies which bind to the modified envelope proteins of the invention. This anti-idiotypic antibody may also be labeled with a detectable marker. Suitable detectable markers are well known to those of ordinary skill in the art and may be, but are not limited to radioisotopes, dyes or enzymes such as peroxidase or alkaline phosphatase.

The anti-idiotypic antibody is produced when an animal is injected with a monoclonal antibody which binds to the modified envelope proteins of the invention. The animal will then produce antibodies directed against the idiotypic determinants of the injected antibody (Wasserman et al. (1982), Proc. Natl. Acad. Sci. 79, 4810-4814).

Alternatively, the anti-idiotypic antibody is produced by contacting lymphoid cells of an animal with an effective-antibody raising amount of the antigen (i.e., the monoclonal antibody which binds to the olgiomeric gp140 proteins of the invention); collecting the resulting lymphoid cells; fusing the collected lymphoid cells with myeloma cells to produce a series of hybridoma cells, each of which produces a monoclonal antibody; screening the series of hybridoma cells to identify those which secrete a monoclonal antibody capable of binding; culturing the resulting hybridoma cell so identified and separately recovering the anti-idiotypic antibody produced by this cell (Cleveland et al. (1983) Nature 305, 56-57). Animals which may be used for the production of anti-idiotypic antibodies in either of the two above-identified methods include, but are not limited to humans, primates, mice, rats, or rabbits.

Another aspect of the present invention provides a monoclonal antibody-producing hybridoma produced by this fusion of a human-mouse myeloma analog and a human antibody-producing cell. In the preferred embodiments, the antibody-producing cell is a human peripheral blood mononuclear cell (PBM), a mitogen stimulated PBM such as a Pokeweed Mitogen (PWM) or a phytohemagglutinin stimulated normal PBM (PHAS) or an Epstein-Barr Virus (EBV) transformed B cell. The human-mouse myeloma analog described above has-an average fusion efficiency for growth of antibody-secreting hybridomas of greater than 1 out of 25,000 fused cells when fused with human PBM, mitogen stimulated PBM and EBV transformed B cells. Especially useful antibody-producing hybridomas of the present invention are those hybridomas which produce monoclonal antibodies specific for the modified gp41 envelope glycoproteins of the invention.

The invention also concerns a method for producing a monoclonal antibody-producing hybridoma which comprises fusing the human-mouse analog with an antibody-producing cell, especially those antibody-producing cells listed hereinabove, and the monoclonal antibody which said hybridoma produces.

The invention further concerns a method of blocking binding of HIV-1 to human cells (both in vitro and in vivo) and a method of preventing infection of human cells by HIV-1 which comprises contacting HIV-1 with an amount of the human monoclonal antibody directed to an epitope in the modified gp41 ectodomain of the gp160, gp140 and gp120 envelope proteins of the invention, effective to block binding of HIV-1 to human cells and preventing infection of human cells by HIV-1.

### Diagnostic Reagents

The modified gp160, gp140, gp120 and/or gp41 envelope proteins of the present invention may be used as diagnostic reagents in immunoassays to detect anti-HIV-1 antibodies, particularly anti-gp41 antibodies. Many HIV-1 immunoassay formats are available. Thus, the following discussion is only illustrative, not inclusive. See generally, however, U.S. Pat. No. 4,753,873 and EP 0161150 and EP 0216191.

Immunoassay protocols may be based, for example, upon composition, direct reaction, or sandwich-type assays. Protocols may also, for example, be heterogeneous and use solid supports, or may be homogeneous and involve immune reactions in solution. Most assays involved the use of labeled antibody or polypeptide. The labels may be, for example, fluorescent, chemiluminescent, radioactive, or dye molecules. Assays which amplify the signals from the probe are also known, examples of such assays are those which utilize biotin and avidin, and enzyme-labeled and mediated immunoassays, such as ELISA assays.

Typically, an immunoassay for anti-HIV-1 antibody will involve selecting and preparing the test sample, such as a biological sample, and then incubating it with a modified gp160, gp140, gp120 and/or gp41 envelope proteins the present invention under conditions that allow antigen-antibody complexes to form. Such conditions are well known in the art. In a heterogeneous format, the protein or peptide is bound to a solid support to facilitate separation of the sample from the polypeptide after incubation. Examples of solid supports that can be used are nitrocellulose, in membrane or microtiter well form, polyvinylchloride, in sheets or microtiter wells, polystyrene latex, in beads or microtiter plates, polyvinlyidine fluoride, diazotized paper, nylon membranes, activated beads, and Protein A beads. Most preferably, Dynatech, Immulon® microtiter plates or 0.25 inch polystyrene beads are used in the heterogeneous format. The solid support is typically washed after separating it from the test sample.

In homogeneous format, on the other hand, the test sample is incubated with the modified gp160, gp140, gp120 and/or gp41 envelope proteins in solution, under conditions that will precipitate any antigen-antibody complexes that are formed, as is known in the art. The precipitated complexes are then separated from the test sample, for example, by centrifugation. The complexes formed comprising anti-HIV antibody are then detected by any number of techniques. Depending on the format, the complexes can be detected with labeled anti-xenogenic immunoglobulin or, if a competitive format is used, by measuring the amount of bound, labeled competing antibody. These and other formats are well known in the art.

Diagnostic probes useful in such assays of the invention include antibodies to the HIV-1 envelope protein. The antibodies to may be either monoclonal or polyclonal, produced using standard techniques well known in the art (See Harlow & Lane (1988), Antibodies: A Laboratory Manual, Cold Spring Harbor Laboratory Press. They can be used to detect HIV-1 envelope protein by specifically binding to the protein and subsequent detection of the antibody-protein complex by ELISA, Western blot or the like. The modified gp160, gp140, gp120 and/or gp41 envelope proteins used to elicit these antibodies can be any of the variants discussed above. Antibodies are also produced from peptide sequences of modified gp160, gp140, gp120 and/or gp41 envelope proteins using standard techniques in the art (Harlow & Lane, supra). Fragments of the monoclonals or the polyclonal antisera which contain the immunologically significant portion can also be prepared.

### EXAMPLES

The following working examples specifically point out preferred embodiments of the present invention, and are not to be construed as limiting in any way the remainder of the disclosure. Other generic configurations will be apparent to one skilled in the art. All references, including U.S. or foreign patents, referred to in this application are herein incorporated by reference in their entirety.

### Example 1

### gp140 Env Constructs

As a model system to explore the possibility of enhancing the elicitation of antibodies with improved broadly reactive and neutralizing activities, a panel of oligomeric gp140 proteins with deleted N-glycosylation sites in the gp41 ectodomain were designed which were examined as immunogens. Using the HIV-1 CM243, Glade E, R5 primary isolate Env as our model, the highly conserved N-glycosylation sites at positions N610, N615, N624, and N636 (FIG. 1) were removed in various combinations by glutamine substitution for asparagine. Four modified gp140 proteins were selected: (N610Q), (N615Q), (N610/615Q) and (N610/615/624/636Q).

These constructs were used in the vaccinia virus expression system to produce milligram amounts of stable, uncleaved oligomeric gp140 proteins. Briefly, the gp140 proteins were produced by infection of monolayers of BS-C-1 cells (ATCC CCL26) in roller bottles (850 cm²) with the appropriate recombinant vaccinia viruses at a multiplicity of infection (MOI) of 5 pfu/cell. Secreted Env proteins were obtained by harvesting the medium (OPTI-MEM I, Gibco, Invitrogen) of expressing cells at thirty to thirty-six hours post vaccinia virus infection. Envelope glycoproteins were purified as previously described (Earl et al. (1994) J. Virol. 68, 3015-3026, Earl et al. (2001) J. Virol. 75, 645-653) using a combination of lentil lectin Sepharose 4B affinity chromatography (Amersham Pharmacia Biotech) and size exclusion chromatography. Briefly, serum-free medium containing Env was clarified by centrifugation at 12,000×g for fifteen minutes at 4° C., then passed over a lentil lectin Sepharose 4B column followed by a wash of two column volumes of phosphate-buffered saline (PBS) containing 20 mM Tris (pH 7.5), 0.3 M NaCl, 0.5% Triton ×100, followed by one column volume of PBS-20 mM Tris (pH 7.5). The bound Env glycoprotein was eluted with 0.5 M methyl mannopyranoside (Sigma) in PBS (pH 7.4), concentrated using Amincon Centriprep concentrators (molecular weight cut-off of 50 kDa) (Millipore) to a final volume of 1 to 2 ml, filter sterilized through a 0.22 μm PVDF low protein binding syringe filter (Millipore) and stored at 4° C. The Env was further separated into oligomeric and monomeric fractions by size exclusion chromatography in PBS, with a HiLoad 16/60 Superdex 200 column (Amersham Pharmacia Biotech). Protein separation was followed by UV absorbance at 280 nm and concurrently plotted on a strip chart. Purified oligomeric gp140 preparations or monomeric gp120 preparations were again concentrated using Centricon concentrators (Millipore), filter-sterilized, aliquoted and stored at −80° C. Env preparations were quantitated using SDS-PAGE and colloidal Coomassie blue staining (Novex) followed by image analysis with NIH Image (version 1.62) by comparison to a previously prepared reference standard of gp120 (strain 111B) purified under identical conditions and quantified by amino acid analysis.

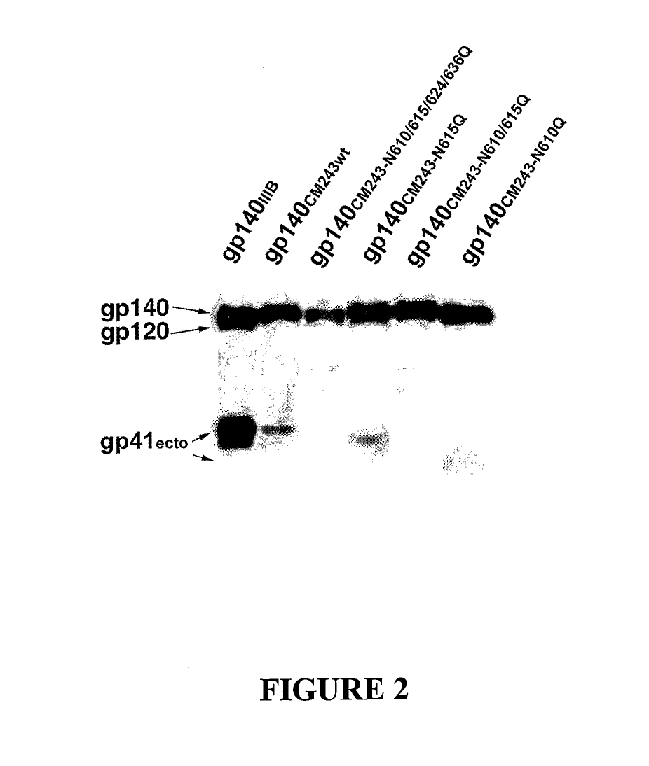

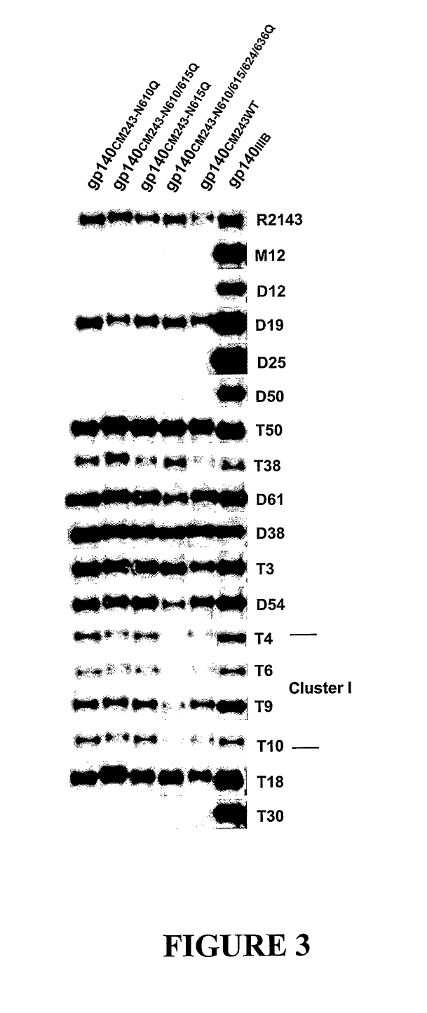

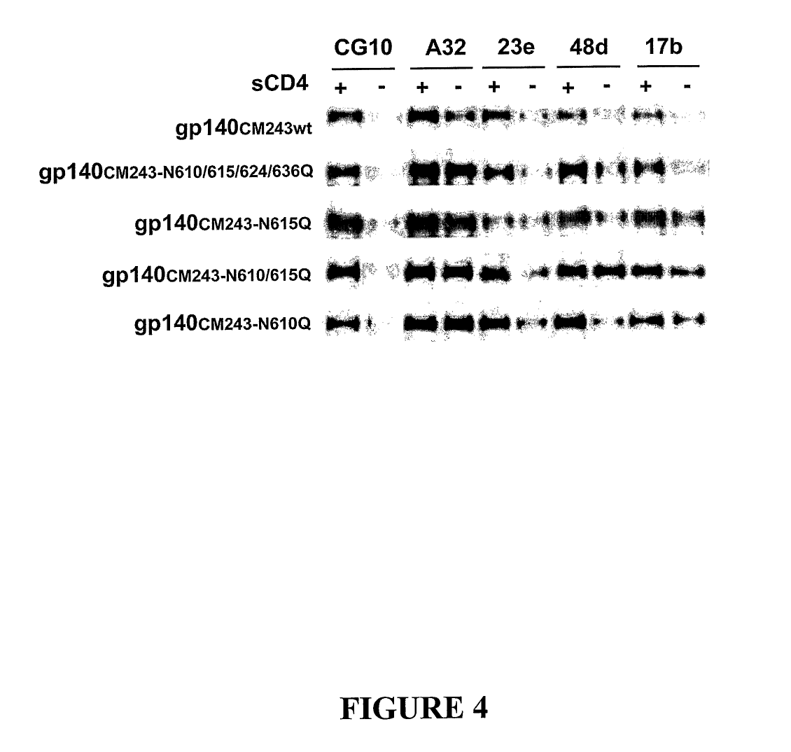

A panel of linear and conformation-dependent, and oligomeric-specific monoclonal antibodies (MAbs) were used to characterize the oligomeric forms of the mutant gp140 panel. Analysis of recombinant expressed and metabolically labeled gp140 proteins by SDS-PAGE revealed that the N610 glycosylation site is indeed modified by carbohydrate (about 5 to 6 kDa) (see FIG. 2). It was also found that mutants possessing the N610Q deletion exhibited better reactivity to the oligomeric-specific monoclonal antibodies (T4, T6, T9, and T10) which map to cluster I, while decreased reactivity to these monoclonal antibodies was seen in the mutant containing a four sites deleted (see FIG. 3). CD4 binding was also retained in the mutant gp140 proteins, as was reactivity with monoclonal antibodies 17b, 48d and 23E following sCD4 binding (CD41 epitopes) indicating the retention of CD4-induced conformational change capacity in the gp140 oligomer. Purified wild type and glycosylation site mutant gp140 (1 μg each) are incubated with or without excess sCD4 (molar ration 1:5) for one hour at room temperature followed by immuoprecipitation by MAb 17b, 48d, 23e and A32. Precipitated gp140 are analyzed by SDS-PAGE and Western with a gp140 reactive polyclonal antiserum 82143 (see FIG. 4).

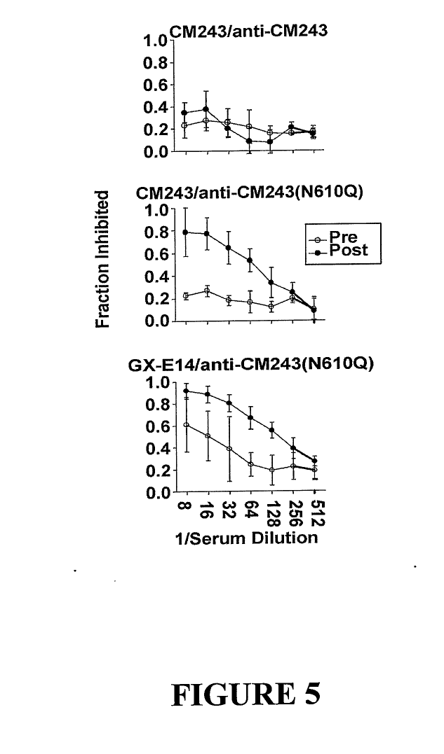

Preparations of wild type and mutant gp140 proteins were then used in rabbit immunization studies. Various soluble oligomeric gp140 preparations were administered to New Zealand White rabbits (Spring Valley Laboratories). Five groups of three animals each were used. Group A received wild type gp140 (gp140_{CM243}), group B received mutant gp140_{CM243-N610/615/624/636Q}, group C received mutant gp140_{CM243-N615Q}, group D received mutant gp140_{CM243-N610/615Q}, group E received mutant gp140_{CM243-N610Q}. Immunizations were given at day 0, 28, 56 and 198. The first immunization consisted of a 100 μg dose of a purified gp140 formulated in 1.0 ml of the MPL+TDM Adjuvant System (Ribi ImmunoChem), consisting of a 2.0% (vol/vol) squaline oil-in-water emulsion containing 250 μg of Monophosphoryl Lipid A (MPL) and 250 μg of Synthetic Trehalose Dicorynomycolate (S-TDCM). The 1.0 ml dose was administered as six 50 μl intradermal sites, 300 μl intramuscular into each hind leg and 100 μl subcutaneous in the neck. Three subsequent immunizations consisted of a 100 μg dose of a purified gp140 formulated with 50 μg QS-21 adjuvant (Antigenics) in 1 ml of phosphate buffered saline and administered by intramuscular injection 0.5 ml into each hind leg. Oligomeric gp140 antigen preparations were vigorously vortexed prior to injection. 1.0 ml dose was administered as 0.05 ml intradermal in six sites, 0.3 ml into each hind leg and 0.1 ml subcutaneous in the neck region. A pre-bleed sample (10 ml) was collected on day 0, a test bleed (10 ml) was collected seven days following the second injection (day 28), a crop bleed sample was collected seven days following the third injection (day 56), and a crop bleed was collected ten days following the forth injection (day 198). All animals were exsangunated at day 349 and serum collected. Each sera was named with the same letter as their group name with numeric number 1, 2 and 3 representing for each individual rabbit (i.e., A1, A2 and A3 for wild type antigen, B1, B2 and B3 for mutant N610/615/6240636Q antigen, C1, C2 and C3 for mutant N615Q antigen, D1, D2 and D3 for mutant N610/615Q antigen, E1, E2 and E3 for mutant N610Q antigen). Alterations of gp41 had a dramatic effect on the neutralizing response induced. The wild-type gp140_{CM243} did not induce detectable neutralizing antibodies, while the N610Q mutation resulted in a marked enhancement of immunogenicity, as shown in FIG. 5.

### Example 2

### Neutralization Assays

The ability of immune sera to inhibit pseudotyped virus infection of HOS-CD4-CCR5 cells, was assayed as described previously (Park et al. (2000) J. Virol. 74, 4183-4191). This assay system has demonstrated that the results obtained are very similar to those obtained using conventional virus neutralization assays of the same primary or laboratory adapted HIV-1 strains (Zhang et al. (1999) J. Virol. 73, 5225-5230). The pseudotyped virus assay is widely employed, rapid, quantitative, and cost-effective. To prepare viruses for these assays, 293T cells were cotransfected with the plasmids pNL4-3.luc.E-R- and pSV7d-HIV-lenv (Deng et al. (1996) Nature 381, 661-666). They included the R2 and SF162 envelope genes and other envelope genes described above. HOS-CD4-CCR5 cell cultures were prepared at approximately 60 to 80% confluency in 96-well opaque walled tissue culture trays, then pretreated with medium containing polybrene (8 μg/ml) for thirty minutes. Serial two-fold serum dilutions were mixed with equal volumes of pseudotyped virus suspensions, and incubated at 4° C. for one hour. The serum-virus mixtures were added to wells of the HOS-CD4-CCR5 cell tissue culture trays and allowed to adsorb for one hour at 37° C. The wells were fed with medium containing polybrene, and incubated at 37° C. in 5% carbon dioxide for two days. The trays were then centrifuged at 1,700 rpm for ten minutes, the cells were washed with PBS, and the trays were drained. Next, 15 μl 1× Luciferase Assay System Cells Lysis Buffer were added per well (Promega). The trays were shaken for thirty minutes at room temperature. Luciferase Assay System Reporter Lysis Buffer (Promega) was added, and luciferase activity was measured using a Luminometer (MicroLumat Plus).

Neutralizing antibody titers were calculated as 50% inhibitory endpoints (Zhang et al. (1999) J. Virol. 73, 5225-5230). Assays were performed in triplicate. Sera from immunized mice were compared to sera from non-immunized control mice maintained in parallel. The 50% inhibition endpoints were determined by comparing the mean luminescence for test samples at each dilution to the mean luminescence for negative control sera. The highest dilution at which the test serum inhibited infectivity to less than 50% of control luminescence was considered the endpoint. In most cases, interpolated 50% inhibitory endpoints were also calculated by regression analysis using Microsoft Excel®. These calculated 50% inhibitory titers corresponded well to titers estimated by comparison of means at each dilution, and the results obtained by comparison of means are presented in this report. The mean luciferase units for the immune sera at 50% neutralization endpoints were consistently significantly lower than the units obtained for the control serum at the same dilution, by Student t test (Excel). Moreover, there was a strong correlation between serum dilution and luciferase units in this assay (Zhang et al. (2002) J. Virol. 76, 644-655). The 50% neutralization endpoints were approximately four- to eight-fold higher dilutions than 90% neutralization endpoints. Since the 50% endpoints were at the middle of the neutralization titration curves, the calculated values were less variable than the 90% endpoints. Also, because of the need to predilute sera substantially before testing (i.e., 1:40), there were some sera that did not achieve 90% neutralization, but mediated statistically significant neutralization >50%. Some inhibition of viral infectivity by control sera was often noted. Percent neutralizing activity in immune sera was determined by comparison of luciferase activity obtained in the presence of immune and control sera. The HNS2 was included for comparison in all assays as a positive control.

### Example 3

### gp140 Env Antibodies

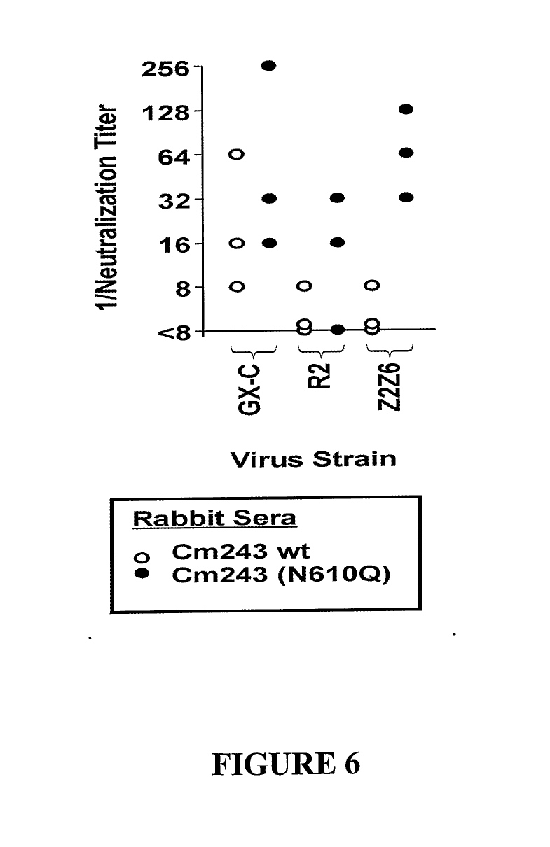

The sera from the rabbits immunized with the gp140_{CM243(N610Q)} also neutralize strains of subtype C, B, and D, as shown in FIG. 6. This cross-reactivity is highly remarkable since sera from donors infected with subtype E strains do not generally neutralize strains of other subtypes at all, particularly subtype B strains. Moreover, the cross-reactivity pattern is much different than we see in response to R2 envelope (a specific HIV-1 Glade B env isolate known to induce cross-reactive neutralizing antibody responses (Dong et al. (2003) J. Virol. 77, 3119-306; Quinnan (1999) AIDS Res Hum Retroviruses 15, 561-70) in which case the sera neutralized subtype A, B, C, and F strains, but not subtype D or E strains. It is also important to recognize that Env-based vaccine strategies to elicit neutralizing antibodies to Glade E viruses have thus far been unsuccessful (Kim et al. (2003) AIDS Res. Hum. Retroviruses 19, 807-816).

In related experiments the CM243 and CM243(N610Q) variants have been compared for neutralization by sera against various gp120 epitopes, as well as 2F5, 4E10 and D61; in the context of full-length env. Viruses pseudotyped with both Envs were resistant to neutralization by the various gp120 monoclonal antibodies tested. Both were highly and equally sensitive to 2F5 and 4E10, and neither was neutralized by D61 (D61 is a mouse monoclonal antibody that defines Cluster I). The comparative neutralization results do not exclude that neutralizing antibodies against any of these epitopes may account for the activity in the sera from the CM243(N610Q) immunized rabbits, but they also do not suggest the mechanism for the remarkable immunogenicity. It is clear that the N610Q mutation has revealed an immunogenicity phenotype that is very rarely found in naturally occurring strains of HIV-1.

### Example 4

### Generation of Immune Response In Vivo

To study the effects of HIV-1 Env protein immunizations in mammals, including primates, administration of the antigen can be accomplished either by DNA expression vectors that produce the desired HIV Env protein or a composition comprising a purified HIV Env protein.

For a DNA expression vaccine, the DNA expression regiment and booster immunizations comprise either modified vaccinia Ankara (MVA) or VEE-RP that express the desired HIV Env protein. Similar regimens have been shown by others to induce potent CD8 T-cell responses (Horton et al. (2002) J. Virol. 76, 7187-7202; McConkey et al. (2003) Nat. Med. 9, 729-735).

For in-vivo expression vectors, VEE-RP-HIV-lenvₑₙᵥ vectors are prepared as described previously, by using pREPX-gp160, pCV, and pGPm as templates for in vitro transcription of RNA (Dong et al. (2003) J. Virol. 77, 3119-3130). VEE-RP-HIV-lenv_{Env} is administered in doses of 10^{6.5} focus forming units (FFU) at weeks 0, 1, 2, 10, 12 and 14 of the study. VEE-RP-SIV env is prepared by cloning of the SIV_{mac251} Env protein (or variant thereof) in pRepX and then processing as for VEE-RP-HIV-lenₑₙᵥ. Dosing includes 10^{6.0} or 10^{7.0} FFU, with half to be given intravenously and half to be given subcutaneously. MVA is prepared as previously described (Horton et at (2002) J. Virol. 76, 7187-7202). The dose of 5×10⁸ PFU in 0.5 ml is administered intradermally in the lateral thigh. The DNA plasmid vaccine VR-SIV env is constructed by inserting a codon optimized SIV env gene into VR1012 vector (Hartikka et al. (1996) Hum. Gen. Ther., 7, 1205-1217). The plasmid is amplified in TOP10 cells (Invitrogen) and by using an endotoxin-free DNA purification kit (Qiagen).

Production of gp140 or derivatives thereof. The gp140 coding sequence is prepared as previously described (Quinnan et al. (1999) AIDS Res. Hum. Retrovir. 14, 939-949). The gene is subcloned into the vaccinia vector p MCO2, linking it to a strong synthetic vaccinia virus early-late promoter (Carroll et al. (1995) Biotechniques 19, 352-354). A recombinant vaccinia virus encoding gp140 (vAC4) is generated by using standard methodology (Broder et al. (1994) Mol. Biotechnol. 13, 223-245). Recombinant gp140 glycoprotein is produced by infecting BS-C-1 cells, and oligomeric gp140 is purified from culture supernatant by using lentil lectin Sepharose 4B affinity and size exclusion chromatography (Earl et al. (1990) J. Viol. 68, 3015-3026; Earl et al. (2001) J. Viol. 75, 645-653). The gp140 is analyzed for binding activity and size.

For initial immunizations, gp140, is prepared in QS-21 adjuvant (Antigenics). Each animal is given 300 μg of gp140 and 150 μg of QS-21 in a total volume of one ml in two divided doses intramuscularly in the hind legs. For the final immunizations, 400 μg of oligomeric gp140 is combined with 1 ml of RiBi adjuvant (Corixa) and then administered in divided doses intramuscularly in the hind legs. Control monkeys receive identical volumes of adjuvant without gp140_{R2}. Although gp140 is cloned, purified and administered in the above example, the same procedure can be followed for any Env protein, including any desired derivatives thereof.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

1. A modified HIV-1 envelope protein or fragment thereof comprising one or more modifications at one or more N-glycosylations sites which, when administered to a mammal, induces the production of a broadly cross-reactive neutralizing anti-serum against multiple subtypes of HIV-1.

2. A modified HIV-1 envelope protein or fragment thereof comprising at least one cross-reactive neutralizing epitope wherein said cross-reactive neutralizing epitope is the result of one or more modifications at an N-glycosylations site on the HIV-1 envelope protein.

3. The modified HIV-1 envelope protein or fragment thereof of claim 1 wherein said modified HIV-1 envelope protein is an oligomeric HIV-1 envelope protein.

4. The modified HIV-1 envelope protein of claim 3 wherein said oligomeric HIV-1 envelope protein is gp140.

5. The modified HIV-1 envelope protein of claim 1 wherein said modified HIV-1 envelope protein is selected from the group consisting of gp160, gp140, gp120 and gp41.

6. The modified HIV-1 envelope protein of claim 1 wherein one or more N-glycosylations sites are deleted.

7. The modified HIV-1 envelope protein of claim 1 wherein one or more N-glycosylations sites are substituted with an amino acid other than asparginine.

8. The modified HIV-1 envelope protein of claim 7 wherein the amino acid other than asparginine is glutamine.

9. The modified oligomeric HIV-1 envelope protein of claim 4 wherein one or more N-glycosylation sites are selected from the group consisting of amino acid corresponding to residues 610, 615, 624 and 636 of SEQ ID NO: 2, 4, 6, 8, 10, 12, 14 and 16.

10. The modified oligomeric HIV-1 envelope protein of claim 4 wherein the envelope protein comprises a sequence selected from the group consisting of SEQ ID NO: 2, 4, 6, 8, 10, 12, 14 and 16.

11. The modified oligomeric HIV-1 envelope protein of claim 10 wherein the envelope protein consists of a sequence selected from the group consisting of SEQ ID NO: 2, 4, 6, 8, 10, 12, 14 and 16.

12. A nucleic acid molecule encoding the modified HIV-1 envelope protein or fragment thereof of claim 1.

13. The nucleic acid molecule of claim 12 wherein the nucleic acid molecule comprises SEQ ID NO: 1, 3, 5, 7, 9, 11, 13 or 15.

14. The nucleic acid molecule of claim 12 wherein the nucleic acid molecule consists of SEQ ID NO: 1, 3, 5, 7, 9, 11, 13 or 15.

15. A nucleic acid molecule having at least about 85%, at least about 90% or at least about 95% sequence identity to any one of the nucleic acid molecules of claim 13.

16. A nucleic acid molecule that specifically hybridizes under stringent hybridization conditions to any one of the nucleic acid molecules of claim 13.

17. The isolated nucleic acid molecule of claim 12 wherein said nucleic acid molecule is operably linked to one or more expression control elements.

18. A vector comprising an isolated nucleic acid molecule of claim 12.

19. A host cell transformed to contain the nucleic acid molecule of claim 12.

20. A host cell comprising the vector of claim 18.

21. The host cell of claim 19, wherein said host is selected from the group consisting of prokaryotic host cells and eukaryotic host cells.

22. A method for producing a polypeptide comprising culturing a host cell transformed with the nucleic acid molecule of claim 12 under conditions in which the polypeptide encoded by said nucleic acid molecule is expressed.

23. A composition comprising the modified HIV-1 envelope protein or fragment thereof of claim 1 and a pharmaceutically acceptable carrier.

24. The composition of claim 23 wherein the composition is suitable as a vaccine in humans.

25. A fusion protein comprising the modified HIV-1 envelope protein or fragment thereof of claim 1.

26. A method of generating antibodies in a mammal comprising administering one or more of the modified HIV-1 envelope proteins or fragments thereof of claim 1 in an amount sufficient to induce the production of the antibodies.

27. A method of generating antibodies in a mammal comprising administering nucleic acids encoding a modified HIV-1 envelope protein or fragment thereof comprising one or more modifications at one or more N-glycosylations sites which, when administered to a mammal, induces the production of broadly cross-reactive neutralizing anti-serum against multiple strains of HIV-1.

28. The method of claim 27 wherein said HIV-1 envelope protein is selected from the group consisting of gp160, gp140, gp120, and gp41.

29. An isolated antibody produced by the method of claim 27.

30. An isolated antibody which specifically binds to any one of the modified HIV-1 envelope proteins or fragments thereof of claim 1.


SEQUENCE LISTING (42 sequences)

SEQ ID NO: 1 - DNA, 2577 bp, Human immunodeficiency virus type 1
  CDS (1)..(2577)
atgagagtgaaggagacacagatgaattggccaaacttgtggaaatgggggactttgatccttgggttggtgataatttgtagtgcctcagacaacttgtgggttacagtttattatggggttcctgtgtggagagatgcagataccaccctattttgtgcatcagatgccaaagcacatgagacggaagtgcacaatgtctgggccacacatgcctgtgtacccacagaccccaacccacaagaaatatacctggaaaatgtaacagaaaattttaacatgtggaacaataacatggtagagcagatgcaggaggatgtaatcagtttatgggatcaaagtctaaagccatgtgtaaagttaactcctctctgcgttactttaatttgtaccaatgctaagttgaccaatgctaatttgaccaatgtcaataacataaccaatgtctctaacataataggaaatataacagatgaagtaagaaactgttcttttaatatgaccacagaactaagagataagaagcagaaggtccatgcacttttttataagcttgatatagtacaaattggagataagaatagtagtgagtataggttaataaattgtaatacttcagtcattaagcaggcttgtccaaagatatcctttgatccaattcctatacattattgtactccagctggttatgcgatttttaagtgtaatgataagaatttcaatgggacagggccatgtaaaaatgtcagctcagtacaatgcacacatggaattaagccagtggtatcaactcaattgctgttaaatggcagtctagcagaagaagagataataatcagatctgaaaatctcacagacaatgccaaaaccataatagtgcaccttaataaatctgtaggaatcaattgtaccagaccctccaacaatacaagrccaagtataactgtrggaccaggacaagtattctatagaacaggagacataataggagatataagragagcatattgtgagattaatggaacaaaatggaatagagttttaaaacaggtaactgaaaaattaaaagagcactttaataataagacaataatctttcaaccaccctcaggaggagatctggaaattacaatgcatcattttaattgtagaggggaatttttctattgcaatacaacacgactgtttaataatacttgcataggaaatgaaaccatgaatgggtgtaatggcactatcacacttccatgcaagataaagcaaattataaacatgtggcagggagcaggacaagcaatgtatgctcctcccatcagtggaaaaattaattgtgtatcaaatattacaggaatactattgacaagagatggtggtgctaatactacgactaacgagaccttcagacctggaggaggaaatataaaggacaattggagaagtgaattatataaatataaagtagtacaaattgaaccactaggaatagcacccaccagggcaaagagaagagtggtggagagagaaaaaagagcagtgggaataggagctatgatctttgggttcttaggagcagcaggaagcactatgggcgcggcgtcaataacgctgacggtacaggccagacaattattgtctggtatagtgcaacagcaragcaatttgctgagggctatwgaggcgcaacagcatctgttgcaactcacagtctggggcatyaarcagctccaggcaagagtcytrgctgtggaaagatacctaaaggatcaaaagctcctrggactttggggytgctctggaaaaatcatctgcaccactgctgtgccctggcagtccacttggagtaatagatcttttgaagagatttggaacaacatgacatggatagaatgggaragagaaattagcaattacacaaaccaaatatatgagatacttacagaatcgcagaaccagcaggacaggaatgaaaaggatttgttagaattggataaatgggcaagcctgtggagttggtttgacataacaaattggctgtggtatataaaaatatttataatgatagtaggaggtttgataggtttaagaataatttttgctgtkctttctatagtgaatagagttaggcagggatactcacctttgtctctccagacccctacccatcatcagagggaactcgacagacccgaaagaatcgaagaaggaggtggcgaacaaggcagagaaagatccgtgcgcttagtgagcggattcttagcacttgcctgggacgatctacggagcctgtgccttttcagctaccaccgcttgagagacttcatctcgattgcagcgagggctgtggaacttctgggacacagcagtctcaagggactaagacgggggtgggaaggcctcaaatatctggggaatcttctgttatattggggccaggaactaaaaattagtgctatttctttgcttaatgctacagcaatagcagtagcggggtggacagataaggttatagaagtagcacaaggagcttggagagccattctccacatacctagaagaatcagacagggcttcgaaagg

SEQ ID NO: 2 - protein, 859 aa, Human immunodeficiency virus type 1
MRVKETQMNWPNLWKWGTLILGLVIICSASDNLWVTVYYGVPVWRDADTTLFCASDAKAHETEVHNVWATHACVPTDPNPQEIYLENVTENFNMWNNNMVEQMQEDVISLWDQSLKPCVKLTPLCVTLICTNAKLTNANLTNVNNITNVSNIIGNITDEVRNCSFNMTTELRDKKQKVHALFYKLDIVQIGDKNSSEYRLINCNTSVIKQACPKISFDPIPIHYCTPAGYAIFKCNDKNFNGTGPCKNVSSVQCTHGIKPVVSTQLLLNGSLAEEEIIIRSENLTDNAKTIIVHLNKSVGINCTRPSNNTRPSITVGPGQVFYRTGDIIGDIRRAYCEINGTKWNRVLKQVTEKLKEHFNNKTIIFQPPSGGDLEITMHHFNCRGEFFYCNTTRLFNNTCIGNETMNGCNGTITLPCKIKQIINMWQGAGQAMYAPPISGKINCVSNITGILLTRDGGANTTTNETFRPGGGNIKDNWRSELYKYKVVQIEPLGIAPTRAKRRVVEREKRAVGIGAMIFGFLGAAGSTMGAASITLTVQARQLLSGIVQQQSNLLRAIEAQQHLLQLTVWGIKQLQARVLAVERYLKDQKLLGLWGCSGKIICTTAVPWQSTWSNRSFEEIWNNMTWIEWEREISNYTNQIYEILTESQNQQDRNEKDLLELDKWASLWSWFDITNWLWYIKIFIMIVGGLIGLRIIFAVLSIVNRVRQGYSPLSLQTPTHHQRELDRPERIEEGGGEQGRERSVRLVSGFLALAWDDLRSLCLFSYHRLRDFISIAARAVELLGHSSLKGLRRGWEGLKYLGNLLLYWGQELKISAISLLNATAIAVAGWTDKVIEVAQGAWRAILHIPRRIRQGFER

SEQ ID NO: 3 - DNA, 2577 bp, Human immunodeficiency virus type 1
  CDS (1)..(2577)
atgagagtgaaggagacacagatgaattggccaaacttgtggaaatgggggactttgatccttgggttggtgataatttgtagtgcctcagacaacttgtgggttacagtttattatggggttcctgtgtggagagatgcagataccaccctattttgtgcatcagatgccaaagcacatgagacggaagtgcacaatgtctgggccacacatgcctgtgtacccacagaccccaacccacaagaaatatacctggaaaatgtaacagaaaattttaacatgtggaacaataacatggtagagcagatgcaggaggatgtaatcagtttatgggatcaaagtctaaagccatgtgtaaagttaactcctctctgcgttactttaatttgtaccaatgctaagttgaccaatgctaatttgaccaatgtcaataacataaccaatgtctctaacataataggaaatataacagatgaagtaagaaactgttcttttaatatgaccacagaactaagagataagaagcagaaggtccatgcacttttttataagcttgatatagtacaaattggagataagaatagtagtgagtataggttaataaattgtaatacttcagtcattaagcaggcttgtccaaagatatcctttgatccaattcctatacattattgtactccagctggttatgcgatttttaagtgtaatgataagaatttcaatgggacagggccatgtaaaaatgtcagctcagtacaatgcacacatggaattaagccagtggtatcaactcaattgctgttaaatggcagtctagcagaagaagagataataatcagatctgaaaatctcacagacaatgccaaaaccataatagtgcaccttaataaatctgtaggaatcaattgtaccagaccctccaacaatacaagrccaagtataactgtrggaccaggacaagtattctatagaacaggagacataataggagatataagragagcatattgtgagattaatggaacaaaatggaatagagttttaaaacaggtaactgaaaaattaaaagagcactttaataataagacaataatctttcaaccaccctcaggaggagatctggaaattacaatgcatcattttaattgtagaggggaatttttctattgcaatacaacacgactgtttaataatacttgcataggaaatgaaaccatgaatgggtgtaatggcactatcacacttccatgcaagataaagcaaattataaacatgtggcagggagcaggacaagcaatgtatgctcctcccatcagtggaaaaattaattgtgtatcaaatattacaggaatactattgacaagagatggtggtgctaatactacgactaacgagaccttcagacctggaggaggaaatataaaggacaattggagaagtgaattatataaatataaagtagtacaaattgaaccactaggaatagcacccaccagggcaaagagaagagtggtggagagagaaaaaagagcagtgggaataggagctatgatctttgggttcttaggagcagcaggaagcactatgggcgcggcgtcaataacgctgacggtacaggccagacaattattgtctggtatagtgcaacagcaragcaatttgctgagggctatwgaggcgcaacagcatctgttgcaactcacagtctggggcatyaarcagctccaggcaagagtcytrgctgtggaaagatacctaaaggatcaaaagctcctrggactttggggytgctctggaaaaatcatctgcaccactgctgtgccctggaactccacttggagtcagagatcttttgaagagatttggaacaacatgacatggatagaatgggaragagaaattagcaattacacaaaccaaatatatgagatacttacagaatcgcagaaccagcaggacaggaatgaaaaggatttgttagaattggataaatgggcaagcctgtggagttggtttgacataacaaattggctgtggtatataaaaatatttataatgatagtaggaggtttgataggtttaagaataatttttgctgtkctttctatagtgaatagagttaggcagggatactcacctttgtctctccagacccctacccatcatcagagggaactcgacagacccgaaagaatcgaagaaggaggtggcgaacaaggcagagaaagatccgtgcgcttagtgagcggattcttagcacttgcctgggacgatctacggagcctgtgccttttcagctaccaccgcttgagagacttcatctcgattgcagcgagggctgtggaacttctgggacacagcagtctcaagggactaagacgggggtgggaaggcctcaaatatctggggaatcttctgttatattggggccaggaactaaaaattagtgctatttctttgcttaatgctacagcaatagcagtagcggggtggacagataaggttatagaagtagcacaaggagcttggagagccattctccacatacctagaagaatcagacagggcttcgaaagg

SEQ ID NO: 4 - protein, 859 aa, Human immunodeficiency virus type 1
MRVKETQMNWPNLWKWGTLILGLVIICSASDNLWVTVYYGVPVWRDADTTLFCASDAKAHETEVHNVWATHACVPTDPNPQEIYLENVTENFNMWNNNMVEQMQEDVISLWDQSLKPCVKLTPLCVTLICTNAKLTNANLTNVNNITNVSNIIGNITDEVRNCSFNMTTELRDKKQKVHALFYKLDIVQIGDKNSSEYRLINCNTSVIKQACPKISFDPIPIHYCTPAGYAIFKCNDKNFNGTGPCKNVSSVQCTHGIKPVVSTQLLLNGSLAEEEIIIRSENLTDNAKTIIVHLNKSVGINCTRPSNNTRPSITVGPGQVFYRTGDIIGDIRRAYCEINGTKWNRVLKQVTEKLKEHFNNKTIIFQPPSGGDLEITMHHFNCRGEFFYCNTTRLFNNTCIGNETMNGCNGTITLPCKIKQIINMWQGAGQAMYAPPISGKINCVSNITGILLTRDGGANTTTNETFRPGGGNIKDNWRSELYKYKVVQIEPLGIAPTRAKRRVVEREKRAVGIGAMIFGFLGAAGSTMGAASITLTVQARQLLSGIVQQQSNLLRAIEAQQHLLQLTVWGIKQLQARVLAVERYLKDQKLLGLWGCSGKIICTTAVPWNSTWSQRSFEEIWNNMTWIEWEREISNYTNQIYEILTESQNQQDRNEKDLLELDKWASLWSWFDITNWLWYIKIFIMIVGGLIGLRIIFAVLSIVNRVRQGYSPLSLQTPTHHQRELDRPERIEEGGGEQGRERSVRLVSGFLALAWDDLRSLCLFSYHRLRDFISIAARAVELLGHSSLKGLRRGWEGLKYLGNLLLYWGQELKISAISLLNATAIAVAGWTDKVIEVAQGAWRAILHIPRRIRQGFER

SEQ ID NO: 5 - DNA, 2577 bp, Human immunodeficiency virus type 1
  CDS (1)..(2577)
atgagagtgaaggagacacagatgaattggccaaacttgtggaaatgggggactttgatccttgggttggtgataatttgtagtgcctcagacaacttgtgggttacagtttattatggggttcctgtgtggagagatgcagataccaccctattttgtgcatcagatgccaaagcacatgagacggaagtgcacaatgtctgggccacacatgcctgtgtacccacagaccccaacccacaagaaatatacctggaaaatgtaacagaaaattttaacatgtggaacaataacatggtagagcagatgcaggaggatgtaatcagtttatgggatcaaagtctaaagccatgtgtaaagttaactcctctctgcgttactttaatttgtaccaatgctaagttgaccaatgctaatttgaccaatgtcaataacataaccaatgtctctaacataataggaaatataacagatgaagtaagaaactgttcttttaatatgaccacagaactaagagataagaagcagaaggtccatgcacttttttataagcttgatatagtacaaattggagataagaatagtagtgagtataggttaataaattgtaatacttcagtcattaagcaggcttgtccaaagatatcctttgatccaattcctatacattattgtactccagctggttatgcgatttttaagtgtaatgataagaatttcaatgggacagggccatgtaaaaatgtcagctcagtacaatgcacacatggaattaagccagtggtatcaactcaattgctgttaaatggcagtctagcagaagaagagataataatcagatctgaaaatctcacagacaatgccaaaaccataatagtgcaccttaataaatctgtaggaatcaattgtaccagaccctccaacaatacaagrccaagtataactgtrggaccaggacaagtattctatagaacaggagacataataggagatataagragagcatattgtgagattaatggaacaaaatggaatagagttttaaaacaggtaactgaaaaattaaaagagcactttaataataagacaataatctttcaaccaccctcaggaggagatctggaaattacaatgcatcattttaattgtagaggggaatttttctattgcaatacaacacgactgtttaataatacttgcataggaaatgaaaccatgaatgggtgtaatggcactatcacacttccatgcaagataaagcaaattataaacatgtggcagggagcaggacaagcaatgtatgctcctcccatcagtggaaaaattaattgtgtatcaaatattacaggaatactattgacaagagatggtggtgctaatactacgactaacgagaccttcagacctggaggaggaaatataaaggacaattggagaagtgaattatataaatataaagtagtacaaattgaaccactaggaatagcacccaccagggcaaagagaagagtggtggagagagaaaaaagagcagtgggaataggagctatgatctttgggttcttaggagcagcaggaagcactatgggcgcggcgtcaataacgctgacggtacaggccagacaattattgtctggtatagtgcaacagcaragcaatttgctgagggctatwgaggcgcaacagcatctgttgcaactcacagtctggggcatyaarcagctccaggcaagagtcytrgctgtggaaagatacctaaaggatcaaaagctcctrggactttggggytgctctggaaaaatcatctgcaccactgctgtgccctggcagtccacttggagtcagagatcttttgaagagatttggaacaacatgacatggatagaatgggaragagaaattagcaattacacaaaccaaatatatgagatacttacagaatcgcagaaccagcaggacaggaatgaaaaggatttgttagaattggataaatgggcaagcctgtggagttggtttgacataacaaattggctgtggtatataaaaatatttataatgatagtaggaggtttgataggtttaagaataatttttgctgtkctttctatagtgaatagagttaggcagggatactcacctttgtctctccagacccctacccatcatcagagggaactcgacagacccgaaagaatcgaagaaggaggtggcgaacaaggcagagaaagatccgtgcgcttagtgagcggattcttagcacttgcctgggacgatctacggagcctgtgccttttcagctaccaccgcttgagagacttcatctcgattgcagcgagggctgtggaacttctgggacacagcagtctcaagggactaagacgggggtgggaaggcctcaaatatctggggaatcttctgttatattggggccaggaactaaaaattagtgctatttctttgcttaatgctacagcaatagcagtagcggggtggacagataaggttatagaagtagcacaaggagcttggagagccattctccacatacctagaagaatcagacagggcttcgaaagg

SEQ ID NO: 6 - protein, 859 aa, Human immunodeficiency virus type 1
MRVKETQMNWPNLWKWGTLILGLVIICSASDNLWVTVYYGVPVWRDADTTLFCASDAKAHETEVHNVWATHACVPTDPNPQEIYLENVTENFNMWNNNMVEQMQEDVISLWDQSLKPCVKLTPLCVTLICTNAKLTNANLTNVNNITNVSNIIGNITDEVRNCSFNMTTELRDKKQKVHALFYKLDIVQIGDKNSSEYRLINCNTSVIKQACPKISFDPIPIHYCTPAGYAIFKCNDKNFNGTGPCKNVSSVQCTHGIKPVVSTQLLLNGSLAEEEIIIRSENLTDNAKTIIVHLNKSVGINCTRPSNNTRPSITVGPGQVFYRTGDIIGDIRRAYCEINGTKWNRVLKQVTEKLKEHFNNKTIIFQPPSGGDLEITMHHFNCRGEFFYCNTTRLFNNTCIGNETMNGCNGTITLPCKIKQIINMWQGAGQAMYAPPISGKINCVSNITGILLTRDGGANTTTNETFRPGGGNIKDNWRSELYKYKVVQIEPLGIAPTRAKRRVVEREKRAVGIGAMIFGFLGAAGSTMGAASITLTVQARQLLSGIVQQQSNLLRAIEAQQHLLQLTVWGIKQLQARVLAVERYLKDQKLLGLWGCSGKIICTTAVPWQSTWSQRSFEEIWNNMTWIEWEREISNYTNQIYEILTESQNQQDRNEKDLLELDKWASLWSWFDITNWLWYIKIFIMIVGGLIGLRIIFAVLSIVNRVRQGYSPLSLQTPTHHQRELDRPERIEEGGGEQGRERSVRLVSGFLALAWDDLRSLCLFSYHRLRDFISIAARAVELLGHSSLKGLRRGWEGLKYLGNLLLYWGQELKISAISLLNATAIAVAGWTDKVIEVAQGAWRAILHIPRRIRQGFER

SEQ ID NO: 7 - DNA, 2577 bp, Human immunodeficiency virus type 1
  CDS (1)..(2577)
atgagagtgaaggagacacagatgaattggccaaacttgtggaaatgggggactttgatccttgggttggtgataatttgtagtgcctcagacaacttgtgggttacagtttattatggggttcctgtgtggagagatgcagataccaccctattttgtgcatcagatgccaaagcacatgagacggaagtgcacaatgtctgggccacacatgcctgtgtacccacagaccccaacccacaagaaatatacctggaaaatgtaacagaaaattttaacatgtggaacaataacatggtagagcagatgcaggaggatgtaatcagtttatgggatcaaagtctaaagccatgtgtaaagttaactcctctctgcgttactttaatttgtaccaatgctaagttgaccaatgctaatttgaccaatgtcaataacataaccaatgtctctaacataataggaaatataacagatgaagtaagaaactgttcttttaatatgaccacagaactaagagataagaagcagaaggtccatgcacttttttataagcttgatatagtacaaattggagataagaatagtagtgagtataggttaataaattgtaatacttcagtcattaagcaggcttgtccaaagatatcctttgatccaattcctatacattattgtactccagctggttatgcgatttttaagtgtaatgataagaatttcaatgggacagggccatgtaaaaatgtcagctcagtacaatgcacacatggaattaagccagtggtatcaactcaattgctgttaaatggcagtctagcagaagaagagataataatcagatctgaaaatctcacagacaatgccaaaaccataatagtgcaccttaataaatctgtaggaatcaattgtaccagaccctccaacaatacaagrccaagtataactgtrggaccaggacaagtattctatagaacaggagacataataggagatataagragagcatattgtgagattaatggaacaaaatggaatagagttttaaaacaggtaactgaaaaattaaaagagcactttaataataagacaataatctttcaaccaccctcaggaggagatctggaaattacaatgcatcattttaattgtagaggggaatttttctattgcaatacaacacgactgtttaataatacttgcataggaaatgaaaccatgaatgggtgtaatggcactatcacacttccatgcaagataaagcaaattataaacatgtggcagggagcaggacaagcaatgtatgctcctcccatcagtggaaaaattaattgtgtatcaaatattacaggaatactattgacaagagatggtggtgctaatactacgactaacgagaccttcagacctggaggaggaaatataaaggacaattggagaagtgaattatataaatataaagtagtacaaattgaaccactaggaatagcacccaccagggcaaagagaagagtggtggagagagaaaaaagagcagtgggaataggagctatgatctttgggttcttaggagcagcaggaagcactatgggcgcggcgtcaataacgctgacggtacaggccagacaattattgtctggtatagtgcaacagcaragcaatttgctgagggctatwgaggcgcaacagcatctgttgcaactcacagtctggggcatyaarcagctccaggcaagagtcytrgctgtggaaagatacctaaaggatcaaaagctcctrggactttggggytgctctggaaaaatcatctgcaccactgctgtgccctggcagtccacttggagtcagagatcttttgaagagatttggaaccagatgacatggatagaatgggaragagaaattagccagtacacaaaccaaatatatgagatacttacagaatcgcagaaccagcaggacaggaatgaaaaggatttgttagaattggataaatgggcaagcctgtggagttggtttgacataacaaattggctgtggtatataaaaatatttataatgatagtaggaggtttgataggtttaagaataatttttgctgtkctttctatagtgaatagagttaggcagggatactcacctttgtctctccagacccctacccatcatcagagggaactcgacagacccgaaagaatcgaagaaggaggtggcgaacaaggcagagaaagatccgtgcgcttagtgagcggattcttagcacttgcctgggacgatctacggagcctgtgccttttcagctaccaccgcttgagagacttcatctcgattgcagcgagggctgtggaacttctgggacacagcagtctcaagggactaagacgggggtgggaaggcctcaaatatctggggaatcttctgttatattggggccaggaactaaaaattagtgctatttctttgcttaatgctacagcaatagcagtagcggggtggacagataaggttatagaagtagcacaaggagcttggagagccattctccacatacctagaagaatcagacagggcttcgaaagg

SEQ ID NO: 8 - protein, 859 aa, Human immunodeficiency virus type 1
MRVKETQMNWPNLWKWGTLILGLVIICSASDNLWVTVYYGVPVWRDADTTLFCASDAKAHETEVHNVWATHACVPTDPNPQEIYLENVTENFNMWNNNMVEQMQEDVISLWDQSLKPCVKLTPLCVTLICTNAKLTNANLTNVNNITNVSNIIGNITDEVRNCSFNMTTELRDKKQKVHALFYKLDIVQIGDKNSSEYRLINCNTSVIKQACPKISFDPIPIHYCTPAGYAIFKCNDKNFNGTGPCKNVSSVQCTHGIKPVVSTQLLLNGSLAEEEIIIRSENLTDNAKTIIVHLNKSVGINCTRPSNNTRPSITVGPGQVFYRTGDIIGDIRRAYCEINGTKWNRVLKQVTEKLKEHFNNKTIIFQPPSGGDLEITMHHFNCRGEFFYCNTTRLFNNTCIGNETMNGCNGTITLPCKIKQIINMWQGAGQAMYAPPISGKINCVSNITGILLTRDGGANTTTNETFRPGGGNIKDNWRSELYKYKVVQIEPLGIAPTRAKRRVVEREKRAVGIGAMIFGFLGAAGSTMGAASITLTVQARQLLSGIVQQQSNLLRAIEAQQHLLQLTVWGIKQLQARVLAVERYLKDQKLLGLWGCSGKIICTTAVPWQSTWSQRSFEEIWNQMTWIEWEREISQYTNQIYEILTESQNQQDRNEKDLLELDKWASLWSWFDITNWLWYIKIFIMIVGGLIGLRIIFAVLSIVNRVRQGYSPLSLQTPTHHQRELDRPERIEEGGGEQGRERSVRLVSGFLALAWDDLRSLCLFSYHRLRDFISIAARAVELLGHSSLKGLRRGWEGLKYLGNLLLYWGQELKISAISLLNATAIAVAGWTDKVIEVAQGAWRAILHIPRRIRQGFER

SEQ ID NO: 9 - DNA, 2049 bp, Human immunodeficiency virus type 1
  CDS (1)..(2049)
atgagagtgaaggagacacagatgaattggccaaacttgtggaaatgggggactttgatccttgggttggtgataatttgtagtgcctcagacaacttgtgggttacagtttattatggggttcctgtgtggagagatgcagataccaccctattttgtgcatcagatgccaaagcacatgagacggaagtgcacaatgtctgggccacacatgcctgtgtacccacagaccccaacccacaagaaatatacctggaaaatgtaacagaaaattttaacatgtggaacaataacatggtagagcagatgcaggaggatgtaatcagtttatgggatcaaagtctaaagccatgtgtaaagttaactcctctctgcgttactttaatttgtaccaatgctaagttgaccaatgctaatttgaccaatgtcaataacataaccaatgtctctaacataataggaaatataacagatgaagtaagaaactgttcttttaatatgaccacagaactaagagataagaagcagaaggtccatgcacttttttataagcttgatatagtacaaattggagataagaatagtagtgagtataggttaataaattgtaatacttcagtcattaagcaggcttgtccaaagatatcctttgatccaattcctatacattattgtactccagctggttatgcgatttttaagtgtaatgataagaatttcaatgggacagggccatgtaaaaatgtcagctcagtacaatgcacacatggaattaagccagtggtatcaactcaattgctgttaaatggcagtctagcagaagaagagataataatcagatctgaaaatctcacagacaatgccaaaaccataatagtgcaccttaataaatctgtaggaatcaattgtaccagaccctccaacaatacaagrccaagtataactgtrggaccaggacaagtattctatagaacaggagacataataggagatataagragagcatattgtgagattaatggaacaaaatggaatagagttttaaaacaggtaactgaaaaattaaaagagcactttaataataagacaataatctttcaaccaccctcaggaggagatctggaaattacaatgcatcattttaattgtagaggggaatttttctattgcaatacaacacgactgtttaataatacttgcataggaaatgaaaccatgaatgggtgtaatggcactatcacacttccatgcaagataaagcaaattataaacatgtggcagggagcaggacaagcaatgtatgctcctcccatcagtggaaaaattaattgtgtatcaaatattacaggaatactattgacaagagatggtggtgctaatactacgactaacgagaccttcagacctggaggaggaaatataaaggacaattggagaagtgaattatataaatataaagtagtacaaattgaaccactaggaatagcacccaccagggcaaagagaagagtggtggagagagaaaaaagagcagtgggaataggagctatgatctttgggttcttaggagcagcaggaagcactatgggcgcggcgtcaataacgctgacggtacaggccagacaattattgtctggtatagtgcaacagcaragcaatttgctgagggctatwgaggcgcaacagcatctgttgcaactcacagtctggggcatyaarcagctccaggcaagagtcytrgctgtggaaagatacctaaaggatcaaaagctcctrggactttggggytgctctggaaaaatcatctgcaccactgctgtgccctggcagtccacttggagtaatagatcttttgaagagatttggaacaacatgacatggatagaatgggaragagaaattagcaattacacaaaccaaatatatgagatacttacagaatcgcagaaccagcaggacaggaatgaaaaggatttgttagaattggataaatgggcaagcctgtggagttggtttgacataacaaattggctgtggtatataaaataa

SEQ ID NO: 10 - protein, 682 aa, Human immunodeficiency virus type 1
MRVKETQMNWPNLWKWGTLILGLVIICSASDNLWVTVYYGVPVWRDADTTLFCASDAKAHETEVHNVWATHACVPTDPNPQEIYLENVTENFNMWNNNMVEQMQEDVISLWDQSLKPCVKLTPLCVTLICTNAKLTNANLTNVNNITNVSNIIGNITDEVRNCSFNMTTELRDKKQKVHALFYKLDIVQIGDKNSSEYRLINCNTSVIKQACPKISFDPIPIHYCTPAGYAIFKCNDKNFNGTGPCKNVSSVQCTHGIKPVVSTQLLLNGSLAEEEIIIRSENLTDNAKTIIVHLNKSVGINCTRPSNNTRPSITVGPGQVFYRTGDIIGDIRRAYCEINGTKWNRVLKQVTEKLKEHFNNKTIIFQPPSGGDLEITMHHFNCRGEFFYCNTTRLFNNTCIGNETMNGCNGTITLPCKIKQIINMWQGAGQAMYAPPISGKINCVSNITGILLTRDGGANTTTNETFRPGGGNIKDNWRSELYKYKVVQIEPLGIAPTRAKRRVVEREKRAVGIGAMIFGFLGAAGSTMGAASITLTVQARQLLSGIVQQQSNLLRAIEAQQHLLQLTVWGIKQLQARVLAVERYLKDQKLLGLWGCSGKIICTTAVPWQSTWSNRSFEEIWNNMTWIEWEREISNYTNQIYEILTESQNQQDRNEKDLLELDKWASLWSWFDITNWLWYIK

SEQ ID NO: 11 - DNA, 2049 bp, Human immunodeficiency virus type 1
  CDS (1)..(2049)
atgagagtgaaggagacacagatgaattggccaaacttgtggaaatgggggactttgatccttgggttggtgataatttgtagtgcctcagacaacttgtgggttacagtttattatggggttcctgtgtggagagatgcagataccaccctattttgtgcatcagatgccaaagcacatgagacggaagtgcacaatgtctgggccacacatgcctgtgtacccacagaccccaacccacaagaaatatacctggaaaatgtaacagaaaattttaacatgtggaacaataacatggtagagcagatgcaggaggatgtaatcagtttatgggatcaaagtctaaagccatgtgtaaagttaactcctctctgcgttactttaatttgtaccaatgctaagttgaccaatgctaatttgaccaatgtcaataacataaccaatgtctctaacataataggaaatataacagatgaagtaagaaactgttcttttaatatgaccacagaactaagagataagaagcagaaggtccatgcacttttttataagcttgatatagtacaaattggagataagaatagtagtgagtataggttaataaattgtaatacttcagtcattaagcaggcttgtccaaagatatcctttgatccaattcctatacattattgtactccagctggttatgcgatttttaagtgtaatgataagaatttcaatgggacagggccatgtaaaaatgtcagctcagtacaatgcacacatggaattaagccagtggtatcaactcaattgctgttaaatggcagtctagcagaagaagagataataatcagatctgaaaatctcacagacaatgccaaaaccataatagtgcaccttaataaatctgtaggaatcaattgtaccagaccctccaacaatacaagrccaagtataactgtrggaccaggacaagtattctatagaacaggagacataataggagatataagragagcatattgtgagattaatggaacaaaatggaatagagttttaaaacaggtaactgaaaaattaaaagagcactttaataataagacaataatctttcaaccaccctcaggaggagatctggaaattacaatgcatcattttaattgtagaggggaatttttctattgcaatacaacacgactgtttaataatacttgcataggaaatgaaaccatgaatgggtgtaatggcactatcacacttccatgcaagataaagcaaattataaacatgtggcagggagcaggacaagcaatgtatgctcctcccatcagtggaaaaattaattgtgtatcaaatattacaggaatactattgacaagagatggtggtgctaatactacgactaacgagaccttcagacctggaggaggaaatataaaggacaattggagaagtgaattatataaatataaagtagtacaaattgaaccactaggaatagcacccaccagggcaaagagaagagtggtggagagagaaaaaagagcagtgggaataggagctatgatctttgggttcttaggagcagcaggaagcactatgggcgcggcgtcaataacgctgacggtacaggccagacaattattgtctggtatagtgcaacagcaragcaatttgctgagggctatwgaggcgcaacagcatctgttgcaactcacagtctggggcatyaarcagctccaggcaagagtcytrgctgtggaaagatacctaaaggatcaaaagctcctrggactttggggytgctctggaaaaatcatctgcaccactgctgtgccctggaactccacttggagtcagagatcttttgaagagatttggaacaacatgacatggatagaatgggaragagaaattagcaattacacaaaccaaatatatgagatacttacagaatcgcagaaccagcaggacaggaatgaaaaggatttgttagaattggataaatgggcaagcctgtggagttggtttgacataacaaattggctgtggtatataaaataa

SEQ ID NO: 12 - protein, 682 aa, Human immunodeficiency virus type 1
MRVKETQMNWPNLWKWGTLILGLVIICSASDNLWVTVYYGVPVWRDADTTLFCASDAKAHETEVHNVWATHACVPTDPNPQEIYLENVTENFNMWNNNMVEQMQEDVISLWDQSLKPCVKLTPLCVTLICTNAKLTNANLTNVNNITNVSNIIGNITDEVRNCSFNMTTELRDKKQKVHALFYKLDIVQIGDKNSSEYRLINCNTSVIKQACPKISFDPIPIHYCTPAGYAIFKCNDKNFNGTGPCKNVSSVQCTHGIKPVVSTQLLLNGSLAEEEIIIRSENLTDNAKTIIVHLNKSVGINCTRPSNNTRPSITVGPGQVFYRTGDIIGDIRRAYCEINGTKWNRVLKQVTEKLKEHFNNKTIIFQPPSGGDLEITMHHFNCRGEFFYCNTTRLFNNTCIGNETMNGCNGTITLPCKIKQIINMWQGAGQAMYAPPISGKINCVSNITGILLTRDGGANTTTNETFRPGGGNIKDNWRSELYKYKVVQIEPLGIAPTRAKRRVVEREKRAVGIGAMIFGFLGAAGSTMGAASITLTVQARQLLSGIVQQQSNLLRAIEAQQHLLQLTVWGIKQLQARVLAVERYLKDQKLLGLWGCSGKIICTTAVPWNSTWSQRSFEEIWNNMTWIEWEREISNYTNQIYEILTESQNQQDRNEKDLLELDKWASLWSWFDITNWLWYIK

SEQ ID NO: 13 - DNA, 2049 bp, Human immunodeficiency virus type 1
  CDS (1)..(2049)
atgagagtgaaggagacacagatgaattggccaaacttgtggaaatgggggactttgatccttgggttggtgataatttgtagtgcctcagacaacttgtgggttacagtttattatggggttcctgtgtggagagatgcagataccaccctattttgtgcatcagatgccaaagcacatgagacggaagtgcacaatgtctgggccacacatgcctgtgtacccacagaccccaacccacaagaaatatacctggaaaatgtaacagaaaattttaacatgtggaacaataacatggtagagcagatgcaggaggatgtaatcagtttatgggatcaaagtctaaagccatgtgtaaagttaactcctctctgcgttactttaatttgtaccaatgctaagttgaccaatgctaatttgaccaatgtcaataacataaccaatgtctctaacataataggaaatataacagatgaagtaagaaactgttcttttaatatgaccacagaactaagagataagaagcagaaggtccatgcacttttttataagcttgatatagtacaaattggagataagaatagtagtgagtataggttaataaattgtaatacttcagtcattaagcaggcttgtccaaagatatcctttgatccaattcctatacattattgtactccagctggttatgcgatttttaagtgtaatgataagaatttcaatgggacagggccatgtaaaaatgtcagctcagtacaatgcacacatggaattaagccagtggtatcaactcaattgctgttaaatggcagtctagcagaagaagagataataatcagatctgaaaatctcacagacaatgccaaaaccataatagtgcaccttaataaatctgtaggaatcaattgtaccagaccctccaacaatacaagrccaagtataactgtrggaccaggacaagtattctatagaacaggagacataataggagatataagragagcatattgtgagattaatggaacaaaatggaatagagttttaaaacaggtaactgaaaaattaaaagagcactttaataataagacaataatctttcaaccaccctcaggaggagatctggaaattacaatgcatcattttaattgtagaggggaatttttctattgcaatacaacacgactgtttaataatacttgcataggaaatgaaaccatgaatgggtgtaatggcactatcacacttccatgcaagataaagcaaattataaacatgtggcagggagcaggacaagcaatgtatgctcctcccatcagtggaaaaattaattgtgtatcaaatattacaggaatactattgacaagagatggtggtgctaatactacgactaacgagaccttcagacctggaggaggaaatataaaggacaattggagaagtgaattatataaatataaagtagtacaaattgaaccactaggaatagcacccaccagggcaaagagaagagtggtggagagagaaaaaagagcagtgggaataggagctatgatctttgggttcttaggagcagcaggaagcactatgggcgcggcgtcaataacgctgacggtacaggccagacaattattgtctggtatagtgcaacagcaragcaatttgctgagggctatwgaggcgcaacagcatctgttgcaactcacagtctggggcatyaarcagctccaggcaagagtcytrgctgtggaaagatacctaaaggatcaaaagctcctrggactttggggytgctctggaaaaatcatctgcaccactgctgtgccctggcagtccacttggagtcagagatcttttgaagagatttggaacaacatgacatggatagaatgggaragagaaattagcaattacacaaaccaaatatatgagatacttacagaatcgcagaaccagcaggacaggaatgaaaaggatttgttagaattggataaatgggcaagcctgtggagttggtttgacataacaaattggctgtggtatataaaataa

SEQ ID NO: 14 - protein, 682 aa, Human immunodeficiency virus type 1
MRVKETQMNWPNLWKWGTLILGLVIICSASDNLWVTVYYGVPVWRDADTTLFCASDAKAHETEVHNVWATHACVPTDPNPQEIYLENVTENFNMWNNNMVEQMQEDVISLWDQSLKPCVKLTPLCVTLICTNAKLTNANLTNVNNITNVSNIIGNITDEVRNCSFNMTTELRDKKQKVHALFYKLDIVQIGDKNSSEYRLINCNTSVIKQACPKISFDPIPIHYCTPAGYAIFKCNDKNFNGTGPCKNVSSVQCTHGIKPVVSTQLLLNGSLAEEEIIIRSENLTDNAKTIIVHLNKSVGINCTRPSNNTRPSITVGPGQVFYRTGDIIGDIRRAYCEINGTKWNRVLKQVTEKLKEHFNNKTIIFQPPSGGDLEITMHHFNCRGEFFYCNTTRLFNNTCIGNETMNGCNGTITLPCKIKQIINMWQGAGQAMYAPPISGKINCVSNITGILLTRDGGANTTTNETFRPGGGNIKDNWRSELYKYKVVQIEPLGIAPTRAKRRVVEREKRAVGIGAMIFGFLGAAGSTMGAASITLTVQARQLLSGIVQQQSNLLRAIEAQQHLLQLTVWGIKQLQARVLAVERYLKDQKLLGLWGCSGKIICTTAVPWQSTWSQRSFEEIWNNMTWIEWEREISNYTNQIYEILTESQNQQDRNEKDLLELDKWASLWSWFDITNWLWYIK

SEQ ID NO: 15 - DNA, 2049 bp, Human immunodeficiency virus type 1
  CDS (1)..(2049)
atgagagtgaaggagacacagatgaattggccaaacttgtggaaatgggggactttgatccttgggttggtgataatttgtagtgcctcagacaacttgtgggttacagtttattatggggttcctgtgtggagagatgcagataccaccctattttgtgcatcagatgccaaagcacatgagacggaagtgcacaatgtctgggccacacatgcctgtgtacccacagaccccaacccacaagaaatatacctggaaaatgtaacagaaaattttaacatgtggaacaataacatggtagagcagatgcaggaggatgtaatcagtttatgggatcaaagtctaaagccatgtgtaaagttaactcctctctgcgttactttaatttgtaccaatgctaagttgaccaatgctaatttgaccaatgtcaataacataaccaatgtctctaacataataggaaatataacagatgaagtaagaaactgttcttttaatatgaccacagaactaagagataagaagcagaaggtccatgcacttttttataagcttgatatagtacaaattggagataagaatagtagtgagtataggttaataaattgtaatacttcagtcattaagcaggcttgtccaaagatatcctttgatccaattcctatacattattgtactccagctggttatgcgatttttaagtgtaatgataagaatttcaatgggacagggccatgtaaaaatgtcagctcagtacaatgcacacatggaattaagccagtggtatcaactcaattgctgttaaatggcagtctagcagaagaagagataataatcagatctgaaaatctcacagacaatgccaaaaccataatagtgcaccttaataaatctgtaggaatcaattgtaccagaccctccaacaatacaagrccaagtataactgtrggaccaggacaagtattctatagaacaggagacataataggagatataagragagcatattgtgagattaatggaacaaaatggaatagagttttaaaacaggtaactgaaaaattaaaagagcactttaataataagacaataatctttcaaccaccctcaggaggagatctggaaattacaatgcatcattttaattgtagaggggaatttttctattgcaatacaacacgactgtttaataatacttgcataggaaatgaaaccatgaatgggtgtaatggcactatcacacttccatgcaagataaagcaaattataaacatgtggcagggagcaggacaagcaatgtatgctcctcccatcagtggaaaaattaattgtgtatcaaatattacaggaatactattgacaagagatggtggtgctaatactacgactaacgagaccttcagacctggaggaggaaatataaaggacaattggagaagtgaattatataaatataaagtagtacaaattgaaccactaggaatagcacccaccagggcaaagagaagagtggtggagagagaaaaaagagcagtgggaataggagctatgatctttgggttcttaggagcagcaggaagcactatgggcgcggcgtcaataacgctgacggtacaggccagacaattattgtctggtatagtgcaacagcaragcaatttgctgagggctatwgaggcgcaacagcatctgttgcaactcacagtctggggcatyaarcagctccaggcaagagtcytrgctgtggaaagatacctaaaggatcaaaagctcctrggactttggggytgctctggaaaaatcatctgcaccactgctgtgccctggcagtccacttggagtcagagatcttttgaagagatttggaaccagatgacatggatagaatgggaragagaaattagccagtacacaaaccaaatatatgagatacttacagaatcgcagaaccagcaggacaggaatgaaaaggatttgttagaattggataaatgggcaagcctgtggagttggtttgacataacaaattggctgtggtatataaaataa

SEQ ID NO: 16 - protein, 682 aa, Human immunodeficiency virus type 1
MRVKETQMNWPNLWKWGTLILGLVIICSASDNLWVTVYYGVPVWRDADTTLFCASDAKAHETEVHNVWATHACVPTDPNPQEIYLENVTENFNMWNNNMVEQMQEDVISLWDQSLKPCVKLTPLCVTLICTNAKLTNANLTNVNNITNVSNIIGNITDEVRNCSFNMTTELRDKKQKVHALFYKLDIVQIGDKNSSEYRLINCNTSVIKQACPKISFDPIPIHYCTPAGYAIFKCNDKNFNGTGPCKNVSSVQCTHGIKPVVSTQLLLNGSLAEEEIIIRSENLTDNAKTIIVHLNKSVGINCTRPSNNTRPSITVGPGQVFYRTGDIIGDIRRAYCEINGTKWNRVLKQVTEKLKEHFNNKTIIFQPPSGGDLEITMHHFNCRGEFFYCNTTRLFNNTCIGNETMNGCNGTITLPCKIKQIINMWQGAGQAMYAPPISGKINCVSNITGILLTRDGGANTTTNETFRPGGGNIKDNWRSELYKYKVVQIEPLGIAPTRAKRRVVEREKRAVGIGAMIFGFLGAAGSTMGAASITLTVQARQLLSGIVQQQSNLLRAIEAQQHLLQLTVWGIKQLQARVLAVERYLKDQKLLGLWGCSGKIICTTAVPWQSTWSQRSFEEIWNQMTWIEWEREISQYTNQIYEILTESQNQQDRNEKDLLELDKWASLWSWFDITNWLWYIK

SEQ ID NO: 17 - protein, 50 aa, Human immunodeficiency virus type 1
GLWGCSGKIICTTAVPWNSTWSNRSFEEIWNNMTWIEWEREISNYTNQIY

SEQ ID NO: 18 - protein, 50 aa, Human immunodeficiency virus type 1
GIWGCSGKLICTTNVPWNSSWSNKSQDEIWDNMTWMEWDKEINNYTDIIY

SEQ ID NO: 19 - protein, 50 aa, Human immunodeficiency virus type 1
GIWGCSGKLICTTNVPWNSSWSNKSQDEIWDNMTWMQWEREISNYTDIIY

SEQ ID NO: 20 - protein, 50 aa, Human immunodeficiency virus type 1
GIWGCSGKLICTTNVPWNSSWSNKSQNEIWDNMTWLQWDKEISNYTHIIY

SEQ ID NO: 21 - protein, 50 aa, Human immunodeficiency virus type 1
GIWGCSGKLICTTNVPWNSSWSNKSQDEIWDNMTWLQWDKEISNYTDIIY

SEQ ID NO: 22 - protein, 50 aa, Human immunodeficiency virus type 1
GIWGCSGKLICATTVPWNSSWSNKTQEEIWNNMTWLQWDKEISNYTNIIY

SEQ ID NO: 23 - protein, 50 aa, Human immunodeficiency virus type 1
GIWGCSGKLICTTAVPWNASWSNKSLDEIWDNMTWMEWEREIDNYTSLIY

SEQ ID NO: 24 - protein, 50 aa, Human immunodeficiency virus type 1
GIWGCSGKLICTTTVPWNASWSNKSLDEIWNNMTWMEWEREIDNYTGLIY

SEQ ID NO: 25 - protein, 50 aa, Human immunodeficiency virus type 1
GIWGCSGKLICTTAVPWNSSWSNKSQEDIWDNMTWMQWDREISNYTDTIY

SEQ ID NO: 26 - protein, 50 aa, Human immunodeficiency virus type 1
GIWGCSGKLICTTAVPWNSSWSNKSQEEIWDNMTWMQWDREISNYTDTIY

SEQ ID NO: 27 - protein, 50 aa, Human immunodeficiency virus type 1
GIWGCSGKHICTTTVPWNSSWSNKSLDEIWNNMTWMEWEREIDNYTGLIY

SEQ ID NO: 28 - protein, 50 aa, Human immunodeficiency virus type 1
GLWGCSGKLICTTNVPWNSSWSNKSQDEIWNNMTWMEWEKEISNYSNIIY

SEQ ID NO: 29 - protein, 50 aa, Human immunodeficiency virus type 1
GIWGCSGKLICTTNVPWNSSWSNKSQDEIWDNMTWMQWEKEISNYTDTIY

SEQ ID NO: 30 - protein, 50 aa, Human immunodeficiency virus type 1
GIWGCSGKLICTTNVPWNTSWSNKSYNEIWDNMTWIEWEREISNYTQQIY

SEQ ID NO: 31 - protein, 50 aa, Human immunodeficiency virus type 1
GIWGCSGKLICTTNVPWNSSWSNKSLDEIWDNMTWMEWDKQINNYTEEIY

SEQ ID NO: 32 - protein, 50 aa, Human immunodeficiency virus type 1; identical to SEQ ID NO: 17

SEQ ID NO: 33 - protein, 50 aa, Human immunodeficiency virus type 1
GIWGCSGKLICTTTVPWNSSWSNKTYNDIWDNMTWLQWDKEISNYTDIIY

SEQ ID NO: 34 - protein, 50 aa, Human immunodeficiency virus type 1
GIWGCSGKLICTTAVPWNTSWSNKSLDEIWNNMTWMEWEREINNYTGLIY

SEQ ID NO: 35 - protein, 50 aa, Human immunodeficiency virus type 1
GIWGCSGKLICTTNVPWNSSWSNKSYNDIWDNMTWLQWDKEINNYTQIIY

SEQ ID NO: 36 - protein, 50 aa, Human immunodeficiency virus type 1
GIWGCSGKLICPTNVPWNASWSNKTYNEIWDNMTWIEWDREINNYTQQIY

SEQ ID NO: 37 - protein, 50 aa, Human immunodeficiency virus type 1
GIWGCSGKLICTTAVPWNSSWSNKSQQEIWDNMTWMQWDKEISNYTNTIY

SEQ ID NO: 38 - protein, 50 aa, Human immunodeficiency virus type 1
GIWGCSGKHICTTNVPWNSSWSNKSLEEIWDNMTWMEWEREIDNYTGLIY

SEQ ID NO: 39 - protein, 50 aa, Human immunodeficiency virus type 1
GIWGCSGKLICTTNVPWNFSWSNKSYDEIWDNMTWIEWEREINNYTQTIY

SEQ ID NO: 40 - protein, 50 aa, Human immunodeficiency virus type 1
GLWGCSGKLICTTNVPWNSSWSNKSQEEIWENMTWMEWEKEINNYSNEIY

SEQ ID NO: 41 - protein, 50 aa, Human immunodeficiency virus type 1
GIWGCSGKLICTTTVPWNASWSNKSLDDIWNNMTWMEWDKEIDNYTGLIY

SEQ ID NO: 42 - protein, 50 aa, Human immunodeficiency virus type 1
  misc_feature (28)..(28): Xaa can be Asp or Glu
NLWGCKGRLICYTSVKWNTTWTKNKDNXIWDNLTWQEWDQQINNISSIIY